(19) United States
(12) Patent Application Publication
(10) Pub. No.: US 2026/0258349 A1
(43) Pub. Date: Sep. 3, 2026

(54) Cultured avocado cells and a food product comprising the same

(71) Applicant: Teknologian tutkimuskeskus VTT Oy, Espoo (FI)

(72) Inventors: Liisa NOHYNEK, Espoo (FI); Suvi HÄKKINEN, Espoo (FI); Heiko RISCHER, Espoo (FI); Anneli RITALA-NURMI, Espoo (FI)

(21) Appl. No.: 19/489,916

(22) PCT Filed: Jun. 12, 2024
(86) PCT No.: PCT/FI2024/050304

§ 371 Date: Dec. 4, 2025

(30) Foreign Application Priority Data
Jun. 13, 2023 (FI) 20230038

(51) Int. Cl. C12N 5/04; A23L 19/00

(52) U.S. Cl. CPC C12N 5/04; A23L 19/09

(57) ABSTRACT

Herein is provided cultured avocado cells that contain at least 15 wt-% proteins, a method for producing said cultured avocado cells, as well as use of said avocado cells in or as a food product.

### TECHNICAL FIELD

The present disclosure generally relates to food products. The disclosure relates particularly, though not exclusively, to food-grade cultured avocado cells and food products comprising the same.

### BACKGROUND

This section illustrates useful background information without admission of any technique described herein representative of the state of the art.

Farming of avocado fruit requires significant resources, such as water, the carbon footprint of avocado farming is considered high, and conventional harvesting methods of avocado fruits may be seen as unsustainable. Furthermore, farming of avocado fruit has been associated even with organized crime.

There is hence a need to provide more sustainable alternatives, from both an environmental and human perspective, for avocado fruit in food products.

### SUMMARY

The appended claims define the scope of protection. Any examples and technical descriptions of apparatuses, products and/or methods in the description and/or figures not covered by the claims are presented not as embodiments of the invention but as examples useful for understanding the invention.

According to a first example aspect, there is provided cultured avocado cells comprising at least 15 wt-% proteins, based on the total dry weight of the cultured avocado cells.

Preferably, the cultured avocado cells are food grade avocado cells for human consumption.

Cultured avocado cells has not been suggested as a food product or food ingredient. Surprisingly, cultured avocado cells are usable in or as a food product, and they may have a beneficial macronutrient profile and/or beneficial fatty acids profile. The present cultured avocado cells have a high protein content, much higher compared to mesocarp of matured avocado fruit, based on respective dry weights. This may provide health benefits and reduce energy content of the cultured avocado cells, especially compared to mesocarp of mature avocado fruit.

According to a second example aspect herein is provided a method for producing cultured avocado cells, the method comprising:
providing avocado cells,
initiating a cell suspension culture by inoculating the provided avocado cells into liquid medium and cultivating the avocado cells in the cell suspension culture for a time sufficient and under conditions sufficient to result in a protein content of at least 15 wt-%, based on the total dry weight of the avocado cells, and
harvesting avocado cells from the cell suspension culture to obtain cultured avocado cells.

Preferably, these cultured avocado cells are cultured avocado cells according to the first example aspect.

The present method allows producing avocado cells in a sustainable manner. The present method is typically more sustainable, both from environmental and human point of view, and faster, compared to farming of avocado fruit. The present method may also require less resources compared to avocado farming, which may allow e.g. cost savings.

According to a further example aspect, herein is provided a food product comprising cultured avocado cells according to the first example aspect, preferably 0.1-100 wt-%, further preferably 0.5-90 wt-%, more preferably 1.0-80 wt-%, even more preferably 5-70 wt-%, most preferably 10-65 wt-% cultured avocado cells according to the first example aspect based on the total weight of the food product.

Although the present cultured avocado cells do not have the appearance and/or texture or structure of avocado fruit mesocarp, they can be used in food products, such as guacamole and/or smoothies, to replace or substitute avocado fruit, especially mashed avocado fruit mesocarp. The present cultured avocado cells may have a pleasant taste with avocado flavour attributes.

Different non-binding example aspects and embodiments have been illustrated in the foregoing. The embodiments in the foregoing are used merely to explain selected aspects or steps that may be utilized in different implementations. Some embodiments may be presented only with reference to certain example aspects. It should be appreciated that corresponding embodiments may apply to other example aspects as well.

### BRIEF DESCRIPTION OF THE FIGURES

Some example embodiments will be described with reference to the accompanying figures, in which:

FIG. 1 shows content of saturated fatty acids, monounsaturated fatty acids, polyunsaturated fatty acids, and sterols in freeze dried, cultured avocado cells according to an example embodiment as a function of cultivation time;

FIG. 2 shows analysed fatty acids contents of dried, cultured avocado cells according to an example embodiment and of freeze dried avocado fruit mesocarp;

FIG. 3 shows analysed sterol contents, as well as analysed contents of tyramine, 2-penadecylfuran, and 2-heptadecadienylfuran, of freeze dried, cultured avocado cells according to an example embodiment and of freeze dried avocado fruit mesocarp;

FIG. 4 shows analysed contents of benzaldehyde, trans-2-nonenal, and 1-octanol of freeze dried, cultured avocado cells according to example embodiments and of freeze dried mesocarp of avocado fruits;

FIG. 5 shows analysed contents of 1-hexanol, 3-hexen-1-ol, tetradecane, and 3-octen-2-one of freeze dried, cultured avocado cells according to example embodiments and of freeze dried mesocarp of avocado fruits;

FIG. 6 shows analysed contents of 2-octenal, 1-octen-3-ol, furfural, and copaene of freeze dried, cultured avocado cells according to example embodiments and of freeze dried mesocarp of avocado fruits;

FIG. 7 shows analysed contents of hexanal, dodecane, 2-hexenal, 2-pentylfuran, 1-pentanol, and 2-heptanal of freeze dried, cultured avocado cells according to example embodiments and of freeze dried mesocarp of avocado fruits;

FIG. 8 shows analysed contents of acetaldehyde, methanol, ethanol, and pentanal of freeze dried, cultured avocado cells according to example embodiments and of freeze dried mesocarp of avocado fruits;

FIG. 9 shows analysed contents of isopropyl myristate, octanoic acid, and 2-methoxy-4-vinylphenol of freeze dried, cultured avocado cells according to example embodiments and of freeze dried mesocarp of avocado fruits;

FIG. 10 shows analysed contents of isopropyl palmitate, 2,4-di-tert-butylphenol, and diethyl phtalate of freeze dried, cultured avocado cells according to example embodiments and of freeze dried mesocarp of avocado fruits;

FIG. 11 shows analysed contents of 2-octenal-2-butyl, 2,4-decadienal, hexanoic acid, benzyl alcohol, and caryophyllene oxide of freeze dried, cultured avocado cells according to example embodiments and of freeze dried mesocarp of avocado fruits;

FIG. 12 shows analysed contents of trans-alpha-bergamotene, and caryophyllene of freeze dried, cultured avocado cells according to example embodiments and of freeze dried mesocarp of avocado fruits;

FIG. 13 shows chromatograms of qualitative catechins and procyanidins analysis of cultured avocado cells according to example embodiments;

FIG. 14 shows a photograph of a food product comprising cultured avocado cells according to an example embodiment;

FIG. 15 shows analysed individual amino acid contents of cultured avocado cells according to example embodiments.

### DETAILED DESCRIPTION

Avocado fruit is typically defined as a berry rich in fatty acids, vitamins, minerals, dietary fibre, and antioxidants. As used herein, avocado or avocado fruit preferably refers to Persea americana, and avocado fruit mesocarp to mesocarp of mature avocado fruit, unless otherwise specified. The development of an avocado fruit takes typically 6-12 months from flowering to mature fruit. The herein described method enables production of cultured avocado cells much faster, even within a few days or a week or two. Also, the present method may be considered more sustainable both from an environmental and from a human perspective compared to farming of avocado fruits.

A mature avocado fruit contains typically 10-20 wt-% lipids, based on total weight of the mesocarp. When calculated based on the total dry weight of the mesocarp, the lipid content of mature avocado fruit may be 60-70 wt-%. Main lipids in mature avocado fruit are triglycerides (TGs), also referred to as triacylglycerols (TAGs) or triacylglycerides. Typically, the lipids of a mature avocado fruit consist substantially of neutral lipids or TAGs (typically about 96 wt-% of total lipids weight), phospholipids, and glycolipids.

Comparing to other fruits and/or berries, the lipids content in avocado fruit is high. The high content of lipids or fats in the avocado fruit mesocarp contributes to a rather high energy content of approximately 198 kcal/100 g or 829 kJ/100 g of mesocarp of mature avocado fruit. As used herein, fats encompass oils.

Typically, mature avocado fruit mesocarp contains, based on the total weight of the mesocarp, about 2 wt-% proteins and ca. 10 wt-% or less carbohydrates, even less than 1 wt-% carbohydrates. Typically, soluble protein content in the mesocarp increases during ripening of the avocado fruit. During early stages of fruit growth carbohydrates content is high (even more than 40 wt-% of the total mesocarp weight), but the carbohydrate content decreases during ripening.

Herein is provided cultured avocado cells comprising at least 15 wt-% proteins, based on the total dry weight of the cultured avocado cells. Preferably, the cultured avocado cells comprise, based on the total dry weight of the cultured avocado cells, proteins 15-30 wt-%, preferably 18-30 wt-%, more preferably 20-30 wt-%, even more preferably 25-30 wt-%.

Several methods for assessing protein content are known. For example, protein content may be determined by total amino acid analysis using UPLC analysis of hydrolysed samples, e.g. as described in Example 5 of the present disclosure, or by Kjeldahl method, for example according to NMKL 6:2003, for example as mentioned in Example 3.1 of the present disclosure. Preferably, in the context of the present disclosure, protein content is determined by total amino acid analysis using UPLC analysis of hydrolysed samples, more preferably as described in Example 5. Although the Kjeldahl method may sometimes be seen as a standard method for assessing protein content in food industry, it might give higher protein contents (“overestimate”) compared to so called direct amino acid analysis methods, such as total amino acid analysis by UPLC analysis of hydrolysed samples.

Preferably, when the protein content is determined by total amino acid analysis using UPLC analysis of hydrolysed samples, especially as defined in Example 5, the cultured avocado cells contain at least 15 wt-%, preferably 15-30 wt-%, further preferably 15-25 wt-%, more preferably 18.0-25.0 wt-%, even more preferably 18.0-22-0 wt-% proteins, based on the total dry weight of the cultured avocado cells. Preferably, when the protein content is determined by Kjeldahl method, especially according to NMKL 6:2003 for example as mentioned in Example 3.1, the cultured avocado cells contain at least 15 wt-%, preferably 15-30 wt-%, more preferably 20-30 wt-%, even more preferably 25-30 wt-% proteins, based on the total dry weight of the cultured avocado cells. In this context, the notion contain a certain amount of proteins refers to the reported protein content as determined by the respective analysis method.

Surprisingly, the present cultured avocado cells contain more proteins than avocado fruit mesocarp, based on respective dry weights. The protein content of the present cultured avocado cells may be even 3-fold or more compared to avocado fruit mesocarp, based on respective dry weights, and as shown in the experimental Examples of the present disclosure. “Based on respective dry weights” refers in the context of this disclosure to comparison of values that are calculated based on the total dry weight of the respective sample (cultured avocado cells, avocado fruit mesocarp) in question. An increased protein content can be seen as providing a beneficial nutritional or macronutrient profile, an enabling use as a plant based or plant derived protein source and/or for boosting protein content for examples in food products. Preferably, the present cultured avocado cells are food grade avocado cells for human consumption.

It was surprisingly found that the present cultured avocado cells may comprise one or more macronutrients (fats or lipids, proteins, and/or carbohydrates) in a different share than in the mesocarp of mature avocado fruit.

Preferably, the present cultured avocado cells comprise more proteins and less lipids than mesocarp of mature avocado fruit. Accordingly, in certain preferred embodiments, the cultured avocado cells comprise, based on the total dry weight of the cultured avocado cells, lipids 0.5-10 wt-%, preferably 1-7 wt-%, more preferably 1.5-6 wt-%, even more preferably 2-5 wt-%. In these embodiments, the protein content of the cultured avocado cells is preferably as defined above. These lipid contents are much lower than typical lipid content of mature avocado fruit. The energy content of the cultured avocado cells is in these embodiments much lower compared to mesocarp of mature avocado fruit. The present cultured avocado cells may hence provide a high-protein, low fat, and low energy alternative to avocado fruit.

The present cultured avocado cells may have higher carbohydrate content compared to the mesocarp of mature avocado fruit. In certain embodiments, the cultured avocado cells comprise 10-65 wt-%, preferably 14-65 wt-%, more preferably 25-64 wt-%, even more preferably 55-64 wt-% carbohydrates, based on the total dry weight of the cultured avocado cells. In these embodiments, the protein content or the lipid content or both of the cultured avocado cells is preferably as defined above. Typically, the higher carbohydrate content contributes to a lower total energy content of the cultured avocado cells compared to mesocarp of mature avocado fruit.

The lipid content and/or carbohydrate content is preferably analyzed as described in Example 3.1 of the present disclosure, accounting for water content,

The lipid composition of the present cultured avocado cells may be considered beneficial nutritionally and/or from a health perspective. When normalised with the total lipid content, the share of polyunsaturated fatty acids (PUFAs) may surprisingly be much higher, even fourfold, compared to the share of PUFAs in mesocarp of mature avocado fruit. PUFAs are generally considered to be particularly desired in food due e.g. to health benefits associated therewith. The share of monounsaturated fatty acids (MUFAs) may be lower and/or the content of saturated fatty acids (SAFAs) may be higher in the present cultured avocado cells compared to the mesocarp of mature avocado fruit, when normalised with the respective total lipid content.

In certain preferred embodiments, the cultured avocado cells comprise, based on the total weight of the lipids in the cultured avocado cells, 30-70 wt-%, preferably 40-65 wt-%, more preferably 50-60 wt-% polyunsaturated fatty acids. Optionally, the cultured avocado cells comprise, based on the total weight of the lipids in the cultured avocado cells, 5-15 wt-%, preferably 8-12 wt-% monounsaturated fatty acids, and/or 20-40 wt-%, preferably 25-40 wt-%, more preferably 30-35 wt-% saturated fatty acids.

The content of monounsaturated oleic acid (C18:1) may be lower in the present cultured avocado cells compared to avocado fruit mesocarp. The main fatty acids in the present cultured avocado cells are typically polyunsaturated linoleic acid (C18:2n-6) and α-linoleic acid (C18:3n-3), the content of linoleic acid (C18:2n-6) typically being higher than that of α-linoleic acid (C18:3n-3), and the content of linoleic acid may be higher in the cultured avocado cells compared to the mesocarp of avocado fruit. In certain embodiments, the cultured avocado cells comprise, based on the total weight of lipids in the cultured avocado cells, 30-55 wt-%, preferably 35-50 wt-%, more preferably 40-50 wt-% linoleic acid (C18:2n-6) and/or 5-15 wt-% or 6-11 wt-% α-linoleic acid (C18:3n-3), and optionally 5-10 wt-% oleic acid (C18:1), and optionally 20-30 wt-% or 20-25 wt-% palmitic acid (C16:0). These values differ from those typical to mesocarp of mature avocado fruit. The cultured avocado cells may be substantially without palmitoleic acid (C16:1 9) content, or the palmitoleic acid (C16:1 9) content may be below detection limit, preferably as determined using direct in situ transesterification and gas chromatography e.g. as described in Glaser et al. 2010 High- Throughput Analysis of Total Plasma Fatty Acid Composition with Direct In Situ Transesterification, PLOS ONE (https://doi.org/10.1371/journal.pone.0012045).

As the total lipids content of the present cultured avocado cells is typically significantly lower compared to the lipid content of mesocarp of mature avocado fruit, certain benefits are obtainable. For example, a high amount of PUFAs may be provided with a lower total lipid or fat content and consequently lower energy content. Preferably, the cultured avocado cells comprise, based on the total dry weight of the cultured avocado cells, polyunsaturated fatty acids at least 5 mg/g, preferably 5-25 mg/g, further preferably 7-20 mg/g, more preferably 10-18 mg/g, even more preferably 10-15 mg/g. Preferably, the cultured avocado cells comprise, based on the total dry weight of the cultured avocado cells, linoleic acid (C18:2n6) at least 5 mg/g, such as 5-20 mg/g, preferably at least 8 mg/g, more preferably 8-15 mg/g, even more preferably 8-10 mg/g, and/or α-linoleic acid (C18:3n3) 1 mg/g or more, preferably 1.5 mg/g or more, more preferably 1-3 mg/g, even more preferably 1.5-2 mg/g. These amounts are surprisingly high considering typical total lipids content of the present cultured avocado cells, preferably as defined in the foregoing. The amount of saturated fatty acids, generally considered less healthy than unsaturated fatty acids, may also be kept at a low level when the total lipid or fat content is low. In certain embodiments, the cultured avocado cells comprise 3-15 mg/g, preferably 5-10 mg/g saturated fatty acids, based on the total dry weight of the cultured avocado cells. Such amounts of saturated fatty acids are in fact much lower compared to SAFAs content in mesocarp of mature avocado fruit, based on respective dry weights, although the share of SAFAs, when normalised to total lipid content, may be higher for the present cultured avocado cells. In certain embodiments, the cultured avocado cells comprise less than 10 mg/g or less than 5 mg/g, preferably 1-3 mg/g, monounsaturated fatty acids, optionally comprising oleic acid (C18:1) less than 5 mg/g, preferably at most 2 mg/g, based on the total dry weight of the cultured avocado cells. Such amounts are much lower than in mesocarp of matured avocado fruit, based on respective dry weights.

The content of free fatty acids (FFAs) may be similar in the cultured avocado cells and in the avocado fruit mesocarp, based on respective dry weights, and the present cultured avocado cells may comprise 0.1-1.0 mg/g, preferably 0.2-0.5 mg/g, FFAs based on the total dry weight of the cultured avocado cells.

Sterols content in the present cultured avocado cells may be higher compared to sterols content in mesocarp of mature avocado fruit. In certain preferred embodiments, the cultured avocado cells comprise 0.5-2.0 mg/g, preferably 0.5-1.5 mg/g, more preferably 0.75-1.25 mg/g sterols, based on the total dry weight of the cultured avocado cells. Especially content of β-sitosterol may be higher in the cultured avocado cells compared to avocado fruit mesocarp. For example, the cultured avocado cells may comprise 0.5-1.0 mg/g β-sitosterol, based on the total dry weight of the cultured avocado cells.

In the context of this disclosure, fatty acid contents are preferably determined according to the method reported by Glaser et al. 2010 High-Throughput Analysis of Total Plasma Fatty Acid Composition with Direct In Situ Transesterification, PLOS ONE (https://doi.org/10.1371/journal.pone.0012045), unless otherwise specified. FAME, FFA and sterol contents, respectively, are in the context of the present disclosure preferably determined according to the method described in Seppsnen-Laakso et al. 2017 UPLC-ELSD Analysis of Algal Lipid Classes and Derivatization of Bound and Free Fatty Acids and Sterols for GC-MS Methods, Biofuels from Algae, vol. 1980, pp. 223-232 (DOI: 10.1007/7651_2017_109).

Compounds in the avocado fruit contributing to the typical flavour of its mesocarp include pentanal, (E)-2-pentenal, ethyl acetate, benzaldehyde, hexanal, and (E)-2-hexenal. Of these, pentanal, (E)-2-pentenal, ethyl acetate, and benzaldehyde are believed to contribute to or provide nutty, fruity flavour typically found pleasant. Hexanal and (E)-2-hexenal are believed to contribute to or provide greasiness and grassy notes to the flavour, which greasy and grassy flavour, although typical for avocado fruit mesocarp, may be found less appealing, even unpleasant.

The present cultured avocado cells may comprise benzaldehyde and/or pentanal, preferably both, providing or contributing to pleasant, fruity and nutty flavour, in amounts that are clearly higher than in mature avocado mesocarp, based on respective dry weights. This may provide a stronger and/or more pleasant taste compared to that of mature avocado fruit mesocarp. Accordingly, in certain preferred embodiments, the cultured avocado cells comprise, based on the total dry weight of the cultured avocado cells, pentanal at least 1.5·10⁸ area/g, preferably from 1.5·10⁸ to 3.5·10⁸ area/g, more preferably from 2.0·10⁸ to 3.0·10⁸ area/g, even more preferably from 2.2·10⁸ to 3.0·10⁸ area/g or from 2.5·10⁸ to 3.0·10⁸ area/g; and/or benzaldehyde more than 6.0·10⁸ area/g, preferably from 6.5·10⁸ to 1.5·10⁹ area/g, further preferably from 7.0·10⁸ to 1.4·10⁹ area/g, more preferably from 8.0·10⁸ to 1.3·10⁹ area/g, even more preferably from 1.0·10⁹ to 1.3·10⁹ area/g, as determined with gas chromatography-mass spectrometry (GC-MS), preferably as described in Example 3.0. The content of pentanal and/or of benzaldehyde may be 1.5-fold or more, even twofold or more, respectively, compared to the respective content in avocado fruit mesocarp, based on respective dry weights.

Although hexanal and (E)-2-hexenal may be considered to provide or contribute to less appealing flavour, they are believed to contribute to taste resembling that of avocado fruit mesocarp. The present cultured avocado cells may comprise hexanal more than avocado fruit mesocarp, based on respective dry weights. In certain embodiments, the cultured avocado cells comprise, based on the total dry weight of the cultured avocado cells, from 3·10⁹ to 6·10⁹ area/g or from 4·10⁹ to 5.5·10⁹ area/g hexanal, as determined with GC-MS, preferably as described in Example 3.0. A higher hexanal content in the cultured avocado cells may in certain embodiments be outweighed by higher content of benzaldehyde and/or pentanal in the cultured avocado cells considered to provide or contribute to pleasant, fruity and nutty flavour, especially if benzaldehyde and/or pentanal, preferably both, are present in the above defined ranges. Typically, the amount of 2-hexenal in the present cultured avocado cells is similar to or slightly higher than that in avocado fruit mesocarp. The present cultured avocado cells may comprise, based on the total dry weight of the cultured avocado cells, about 0.5·10⁹ area/g or less 2-hexenal, as determined with GC-MS, preferably as described in Example 3.0. A stronger taste of the present cultured avocado cells compared to mature avocado fruit mesocarp may be beneficial in that a smaller amount of the cultured avocado cells is needed to provide desired avocado like flavour. This may (further) reduce consumption of resources, energy content of the food product, and/or may decrease costs. It has surprisingly been found that fresh cultured avocado cells as described herein have a pleasant flavour and odour, and avocado flavour attributes, as reported in Example 6 of the present disclosure.

The mesocarp of avocado fruit contains various organic compounds referred herein to as volatiles. Many of the volatiles found in the mesocarp of mature avocado fruit are also present in the herein described cultured avocado cells. However, the volatiles and their contents are typically by no means identical between avocado fruit mesocarp and the present cultured avocado cells.

In certain embodiments, the present cultured avocado cells may be substantially without caryophyllene content or their caryophyllene content may be below detection limit, as determined with GC-MS, preferably as described in Example 3.0. Caryophyllene is considered a typical volatile of avocado fruit mesocarp, and hence absence or just very low amounts of it in the cultured avocado cells is surprising. Similarly, the content of tocopherol or vitamin E in the cultured avocado cells may be below detection limit, as determined with GC-MS, preferably as described in Example 3.0, or the cultured avocado cells may be substantially without tocopherol content. Tocopherol is typically present in avocado mesocarp in non-negligible amounts and hence it is surprising that it may be present in just very minor amounts or even absent in the present cultured avocado cells. Accordingly, in certain embodiments, the cultured avocado cells are substantially free from caryophyllene and/or tocopherol, preferably substantially free from caryophyllene and tocopherol.

The present cultured avocado cells may be substantially free from one or more, or even all, of 2-pentadecylfuran, 2-heptadecadienylfuran, 3-hexen-1-ol, caryophyllene oxide, and/or trans-alpha-bergamotene. These are typically found in mesocarp of mature avocado fruits.

The content of one or more of dodecane, 2-heptenal, acetaldehyde, 1-hexanol, tetradecane, furfural, copaene, 1-octanol, and/or 2-heptadecadienylfuran, or all of these, may be lower in the cultured avocado cells compared to the mesocarp of matured avocado fruit, based on respective dry weights. For example, 1-octanol may provide or contribute to unpleasant flavor, and hence a reduced amount may be favoured.

In certain embodiments, the cultured avocado cells comprise, based on the total dry weight of the cultured avocado cells, less than 2·10⁸ area/g, preferably 0.1·10⁸-1.5·10⁸, more preferably 0.5·10⁸-1.0·10⁸ area/g acetaldehyde, as determined with GC-MS, preferably as described in Example 3.0. The acetaldehyde content of the cultured avocado cells may be less than half, or two fifths or less, compared to avocado fruit mesocarp, based on respective dry weights.

In certain embodiments, the cultured avocado cells comprise, based on the total dry weight of the cultured avocado cells, less than 1.0-3.5·10⁸ area/g, preferably 1.5·10⁸-3.0·10⁸ area/g 1-octanol, as determined with GC-MS, preferably as described in Example 3.0. The 1-octanol content of the cultured avocado cells may be ⅔ or less, compared to avocado fruit mesocarp, based on respective dry weights.

In certain embodiments, the cultured avocado cells comprise, based on the total dry weight of the cultured avocado cells, less than 0.5·10⁹ area/g, preferably 0.1·10⁹-1.0·10⁹, more preferably 0.1·10⁹-0.5·10⁹ area/g, dodecane, as determined with GC-MS, preferably as described in Example 3.0. The cultured avocado cells may contain dodecane a fifth or less, even a tenth or less, compared to avocado fruit mesocarp, based on respective dry weights.

In certain embodiments, the cultured avocado cells comprise, based on the total dry weight of the cultured avocado cells, 1·10⁸-3·10⁸ area/g, preferably 1.5·10⁸-2.5·10⁸ area/g 1-hexanol, as determined with GC-MS, preferably as described in Example 3.0. The present cultured avocado cells may comprise 1-hexanol in an amount that is half of or less, even a third of or less, the amount of 1-hexanol in mesocarp of matured avocado fruit, based on respective dry weights.

In certain embodiments, the cultured avocado cells comprise, based on the total dry weight of the cultured avocado cells, 0.5·10⁹-1.3·10⁹ area/g 2-heptenal, as determined with GC-MS, preferably as described in Example 3.0. The present cultured avocado cells may comprise 2-heptenal in an amount that is half of or less, even a third of or less, the amount of 2-heptenal in mesocarp of matured avocado fruit, based on respective dry weights.

In certain embodiments, the cultured avocado cells comprise, based on the total dry weight of the cultured avocado cells, 0.5·10⁸-1.5·10⁸ area/g, preferably 0.7·10⁸-1.5·10⁸ area/g tetradecane, and/or 1.5·10⁸-2.5·10⁸ area/g 3-octen-2-one, as determined with GC-MS, preferably as described in Example 3.0.

In certain embodiments, the cultured avocado cells comprise, based on the total dry weight of the cultured avocado cells, 0.5·10⁸-2·10⁸ area/g, preferably 0.7·10⁸-1.5·10⁸ area/g, more preferably 0.7·10⁸-1.2·10⁸ area/g furfural, as determined with GC-MS, preferably as described in Example 3.0. The furfural content of the cultured avocado cells may be less than a half of the furfural content of avocado fruit mesocarp, even about a fifth or less, based on respective dry weights. The copaene content of the cultured avocado cells may be a fifth or less compared to cultured avocado cells, based on respective dry weights, for example 5·10⁸ area/g or less, as determined with GC-MS, preferably as described in Example 3.0, and based on the total dry weight of the cultured avocado cells.

The content of one or more of tyramine, ethanol, 2-pentylfuran, 2-octenal, 1-octen-3-ol, trans-2-nonenal, isopropyl myristate, octanoic acid, isopropyl palmitate, 2,4-di-tert-butylphenol, hexanoic acid, 2-octanal-2-butyl, and/or benzyl alcohol, or all of these, may be higher in the cultured avocado cells compared to the mesocarp of matured avocado fruit, based on respective dry weights. Trans-2-nonenal may provide slight oily fried taste with nutty background, and its content may be slightly higher in the cultured cells compared to avocado fruit mesocarp, based on respective dry weights.

In certain embodiments, the cultured avocado cells comprise, based on the total dry weight of the cultured avocado cells, 0.8·10⁹ or more area/g, preferably 0.9·10⁹-2·10⁹ area/g, more preferably 1.0·10⁹-1.5·10⁹ area/g 2-octenal, as determined with GC-MS, preferably as described in Example 3.0. The cultured avocado cells may comprise 2-octenal at least 1.5-fold, even 2-fold or more, compared to mesocarp of cultured avocado cells, based on respective dry weights. 2-octenal may provide sweet, fatty, citrus peel like flavour, and such taste notes may be stronger when the content of 2-octenal is higher.

Preferably, the cultured avocado cells comprise catechin(s) and/or procyanidin(s), preferably catechin(s) and/or oligomeric procyanidin(s). Catechin(s) and procyanidin(s), especially oligomeric procyanidin(s), are typically considered to be healthy or provide health benefits.

In certain embodiments, the cultured avocado cells comprise, based on the total dry weight of the cultured avocado cells, 3.5·10⁸-10·10⁸ area/g, preferably 5.0·10⁹-9.0·10⁹ area/g octanoic acid, and/or 0.5·10⁸-3.0·10⁸ area/g, preferably 1.0·10⁸-2.0·10⁸ area/g, isopropyl myristate, as determined with GC-MS, preferably as described in Example 3.0. The octanoic acid content and/or isopropyl myristate content may be 15-fold or higher, even 20-fold or higher, respectively, compared to avocado fruit mesocarp, based on respective dry weights.

In certain embodiments, the cultured avocado cells comprise, based on the total dry weight of the cultured avocado cells, 0.5·10⁹-2·10⁹ area/g, preferably 0.8·10⁹-1.7·10⁹ area/g, more preferably 1.0·10⁹-1.5·10⁹ isopropyl palmitate, as determined with GC-MS, preferably as described in Example 3.0. The cultured avocado cells may comprise isopropyl palmitate at least 5-fold, even 7-fold or more, compared to mesocarp of cultured avocado cells, based on respective dry weights.

In certain embodiments, the cultured avocado cells comprise, based on the total dry weight of the cultured avocado cells, 0.5·10⁹-2·10⁹ area/g, preferably 0.7·10⁹-1.7·10⁹ area/g, more preferably 0.8·10⁹-1.5·10⁹ 2,4-di-tert-butylphenol, as determined with GC-MS, preferably as described in Example 3.0. The cultured avocado cells may comprise 2,4-di-tert-butylphenol at least 8-fold, even 9-fold or more, compared to mesocarp of cultured avocado cells, based on respective dry weights.

In certain embodiments, the cultured avocado cells comprise, based on the total dry weight of the cultured avocado cells, 0.5·10⁹-2.5·10⁹ area/g, preferably 1.0·10⁹-2.0·10⁹ area/g benzyl alcohol, as determined with GC-MS, preferably as described in Example 3.0. The cultured avocado cells may comprise benzyl alcohol at least 50-fold, even 55-fold or more, compared to mesocarp of cultured avocado cells, based on respective dry weights.

The cultured avocado cells may comprise, based on the total dry weight of the cultured avocado cells, 2-octenal-2-butyl 0.3·10⁸ or more area/g, such as 0.5·10⁸-1.5·10⁸ area/g, as determined with GC-MS, preferably as described in Example 3.0. Avocado fruit mesocarp does typically not contain 2-octenal-2-butyl, at least not in significant amounts.

The cultured avocado cells may comprise, based on the total dry weight of the cultured avocado cells, hexanoic acid 5·10⁸-10·10⁸ area/g, as determined with GC-MS, preferably as described in Example 3.0. This is more than typically in avocado fruit mesocarp, based on respective dry weights. Hexanoic acid may contribute to desired flavour of the cultured avocado cells.

As illustrated in the foregoing, the content of certain volatile(s) may differ significantly in the cultured avocado cells compared to avocado fruit mesocarp, based on respective dry weights. The present cultured avocado cells may provide certain compound(s) in desired amount(s) and/or amounts not obtainable by avocado fruit mesocarp.

Herein is provided a method for producing cultured avocado cells, the method comprising: providing avocado cells, initiating a cell suspension culture by inoculating the provided avocado cells into liquid medium and cultivating the avocado cells in the cell suspension culture for a time sufficient and under conditions sufficient to result in a protein content of at least 15 wt-%, based on the total dry weight of the avocado cells, and harvesting avocado cells from the cell suspension culture to obtain cultured avocado cells.

Preferably, the method is for producing the cultured avocado cells described herein, preferably as described in any one of the embodiments described in the foregoing. The cultivation of the avocado cells may be carried out in bioreactor(s).

Preferably, providing avocado cells comprises growing callus from avocado on solid growth medium, and providing avocado cells from the callus (and the cell suspension culture is then initiated from these provided cells). The provided cells may be provided fresh from the callus, or they may be stored for example in liquid nitrogen at temperature below −190° C., before providing them and initiating the cell suspension culture. Typically, frozen cells are thawed before initiating suspension culture.

Callus may be grown from avocado fruit material or from cuts of avocado plant, such as in vitro grown avocado plant(s). In certain embodiments, the callus is grown from avocado seed or seed cut(s), preferably from mature avocado fruit (e.g. 6-12 months from flowering). Preferably, the seed material may be the embryo or portion(s) thereof and/or the seed may have been subjected to germination, typically in water, prior to growing callus therefrom. Similarly, the seed cut(s) may optionally be provided by cutting a seed having been subjected to germination in water. The germination time may be for example from 1 to 3 months, preferably about 2 months. In certain embodiments, the callus is grown from cuts of avocado plant(s), including plantlets, such as cuts of sterile stem. Callus may in certain embodiments be grown from material from inner layer of avocado fruit between fruit cover and mesocarp.

In certain preferred embodiments, the present method comprises sterilizing, preferably surface sterilizing, the portion(s) of the avocado from which callus is grown, prior to growing callus therefrom. Suitable sterilization methods include for example surface sterilization with 70% ethanol, surfactant (e.g. Tween80), and/or sodium hypochlorite (1-10% v/v), followed by washing with sterile water. However, any suitable sterilization or surface sterilization method may be used.

Typically, the callus is grown from avocado, preferably avocado fruit material, plant cut(s), and/or cut(s) from seed(s) as defined above, on solid growth medium. The solid growth medium may comprise plant growth regulators, preferably at least one cytokinin or cytokinin-like plant growth regulator (CN) and at least one auxin (AUX). Examples of suitable CNs and examples of suitable AUXs are mentioned below.

Establishing or growing callus from avocado typically comprises incubating a portion of avocado, preferably seed or seed cut(s) as described above, on solid medium with nutrients and plant growth regulators. For example, the solid medium may be Murashige and Skoog medium (MS-medium, as described in Murashige, T., and Skoog, F. (1962). A revised medium for rapid growth and bioassays with tobacco tissue cultures, Physiol. Plant. 15, 473-497) solidified with a suitable solidification agent, such as agar or Gelrite®. For example, the growth medium may be solidified with 6.5 g/l agar or 3 g/l Gelrite®, but any suitable solidification agent in a suitable amount may be used. Impurities may hamper growth, and hence typically sufficiently pure solidification agent(s) is used.

The solid medium may contain sucrose, for example 2-3% w/v, preferably about 3% w/v. The plant growth regulators may comprise at least one cytokinin or cytokinin-like plant growth regulator (CN) and at least one auxin (AUX). The wt:wt ratio of CN to AUX may be within a range from 1:100 to 1:2. The CN concentration may be 0.1-5.0 ppm and the AUX concentration may be 1.0-10 ppm. Examples of suitable CNs include kinetin, 6-(γ,γ-dimethylallylamino)purine, zeatin, or any combination thereof, and examples of suitable AUXs include a-naphthalene acetic acid, indole acetic acid, picloram, or any combination thereof, preferably a-naphthalene acetic acid, indole acetic acid, or any combination thereof. More preferably, the CN is kinetin preferably in an amount of from 0.1 to 5.0 ppm, more preferably about 0.1 ppm, and the AUX is a-naphthalene acetic acid (NAA) preferably in an amount from 1.0 to 10 ppm, more preferably about 1.0 ppm.

The incubation temperature for establishing the callus may be within a range from 17 to 28° C., for example 22±2° C. The incubation may be performed in dark and/or light, typically the incubation comprises incubation both in dark and in light, such as alternating light and dark periods according to a predetermined regime. Preferably, a day:night photoperiod of 16 h light:8 h darkness is applied, preferably using normal light with irradiation 30-60 μmol/m²s for the day or light periods.

Obtained, soft callus may be transferred to fresh medium. Preferably, the fresh medium is a solid medium with plant growth regulators or nutrients and plant growth regulators as described above. The obtained callus may be maintained and cultivated using a solid medium with plant growth regulators or nutrients and plant growth regulators as described above, and preferably an illumination regime as described above. Optionally, the callus can be maintained in light (without dark periods) or in dark (without light periods). The callus may be maintained at a temperature within a range from 17 to 28° C., such as 24° C. The temperature at which the callus is maintained may be substantially same at which it was established, or it may be different.

Preferably, the callus culture is sub-cultured every 3 to 5 weeks. The subculturing conditions may be as described above for establishing or growing callus.

Providing avocado cells may comprise selecting avocado cells preferably from callus, the callus preferably being obtained as described above. By suitably selecting the cells from which cell suspension culture is initiated, and/or the cell suspension culture conditions and/or duration, desired contents of desired compounds in the cultured cells may be obtained, while presence of certain compounds in the cultured cells may optionally be limited or avoided. As the person skilled in the art appreciates, cultured cells are typically different in their composition and/or properties compared e,g, to naturally occurring fruit or berry cells.

Typically, avocado cells, especially from callus preferably obtained as described above, produce macronutrients (proteins, lipids or fats, carbohydrates) in cell suspension culture.

However, if needed or desired, a selection step of selecting avocado cells that in cell suspension culture produce proteins, lipids, and/or carbohydrates, preferably at least proteins, more preferably proteins, lipids, and carbohydrates, said avocado cells preferably being selected from callus preferably obtained as described in the foregoing, may be comprised in the present method.

In certain preferred embodiments, providing avocado cells comprises
selecting avocado cells that produce catechin(s) and/or procyanidin(s), preferably catechin(s) and/or oligomeric procyanidin(s), more preferably both, in cell suspension culture, and
providing the selected avocado cells (and initiating cell suspension culture from these cells). Catechin(s) and/or procyanidin(s), especially oligomeric procyanidin(s), may provide health benefits.

In certain preferred embodiments, providing avocado cells comprises
selecting avocado cells that produce benzaldehyde and/or pentanal, preferably both, and optionally hexanal and/or (E)-2-hexenal, in cell suspension culture, and
providing the selected avocado cells (and initiating cell suspension culture from these cells). This may provide taste benefits, as described in the foregoing.

In certain preferred embodiments, providing avocado cells comprises
selecting avocado cells that in cell suspension culture are essentially free from one or more, or even all, of 2-pentadecylfuran, 2-heptadecadienylfuran, 3-hexen-1-ol, caryophyllene oxide, trans-alpha-bergamotene, caryophyllene, and/or tocopherol, preferably free from at least caryophyllene and/or tocopherol, more preferably at least both, said avocado cells preferably being selected from callus preferably obtained as described in the foregoing, and
providing the selected avocado cells (and initiating cell suspension culture from these cells).

In certain preferred embodiments, providing avocado cells comprises selecting avocado cells that produce polyunsaturated fatty acids, preferably at least linoleic acid (C18:2n-6) and/or α-linoleic acid (C18:3n-3), preferably both, optionally without substantially producing palmitoleic acid (C16:1 9), in cell suspension culture, said avocado cells preferably being selected from callus preferably obtained as described above, and
providing the selected avocado cells (and initiating cell suspension culture from these cells).

In certain preferred embodiments, providing avocado cells comprises selecting avocado cells that produce sterols, preferably at least β-sitosterol, in cell suspension culture, said avocado cells preferably being selected from callus, preferably obtained as described above, and
providing the selected avocado cells (and initiating cell suspension culture from these cells).

Selecting cells that do produce certain compound(s) in cell suspension culture allows obtaining cultured cells containing these compound(s), possibly in amount(s) that are higher than in mesocarp of avocado fruit. Selecting cells that do substantially not produce certain compound(s) or are substantially free from certain compound(s) in cell suspension culture allows obtaining cultured cells that are naturally substantially without such compound(s), or containing such compound(s) in amounts e.g. below detection limit. “Naturally without” means herein that no removal step of said compound(s) is necessary in order to obtain cultured cells substantially without these compounds.

Cells selected as described above may in cell suspension culture produce cells that differ even significantly in their composition from avocado fruit mesocarp.

It is particularly preferred that the provided avocado cells from which the cell suspension culture is initiated are selected so that they produce and are essentially free from compounds as defined in the combination of the above embodiments.

Preferably, the cultured avocado cells of the present disclosure are obtainable or obtained with the method of the present disclosure or according to any one of the embodiments thereof.

In the present method, cell suspension culture is initiated by inoculating the provided avocado cells, preferably from callus, the callus preferably being obtained as described in the foregoing, and optionally the cells suitably selected as described in the foregoing, into liquid medium, and cultured avocado cells are obtained by harvesting avocado cells from the cell suspension culture. In the present method, the avocado cells are cultivated in the cell suspension culture for a time sufficient and under conditions sufficient to result in a protein content of at least 15 wt-%, preferably 15-30 wt-%, further preferably 18-30 wt-%, more preferably 20-30 wt-%, even more preferably 25-30 wt-%, based on the total dry weight of the avocado cells.

It has surprisingly been found that the protein content of the cultured avocado cells may be further increased by including in the liquid medium glycine (Gly) and glutamic acid (Glu) preferably in a total amount of 0.6-5.0 mM (millimole/dm³), preferably of 0.8-3.0 mM, more preferably of 1.0-2.0 mM. Preferably, Glu is present in the liquid medium in a higher amount than Gly. In certain preferred embodiments, the liquid medium comprises 0.01-0.05 mM Gly and 0.5-1.5 mM Glu.

As discussed in the foregoing, preferably, when the protein content is determined by total amino acid analysis using UPLC analysis of hydrolysed samples, especially as defined in Example 5, the cultured avocado cells contain at least 15 wt-%, preferably 15-30 wt-%, further preferably 15-25 wt-%, more preferably 18.0-25.0 wt-%, even more preferably 18.0-22.0 wt-% proteins, based on the total dry weight of the cultured avocado cells, and preferably, when the protein content is determined by Kjeldahl method, especially according to NMKL 6:2003 for example as mentioned in Example 3.1, the cultured avocado cells contain at least 15-30 wt-%, preferably 20-30 wt-%, even more preferably 25-30 wt-% proteins, based on the total dry weight of the cultured avocado cells.

Cell suspension culture conditions, such as temperature, agitation, culture media, nutrients and their amounts and addition regimes, elicitors and their amounts and addition regimes (as well as lack of elicitor(s)), light and/or dark periods, etc. typically affect production of compounds in the cultivated cells and may reduce or increase production of certain compound(s), as is appreciated by the person skilled in the art. Duration of the cultivation may affect concentrations of at least certain compounds in the cultured cell, as appreciated by the person skilled in the art. Relationship between cultivation time and concentration of a certain compound is not necessary linear, and the concentration of a certain compound may peak at a certain cultivation time range, after which its concentration may start to decrease or become more or less constant. The person skilled in the art appreciates that conditions and/or duration of the cell suspension culture may be suitably selected to result in cultured avocado cells of the present disclosure and/or any embodiment thereof, especially when the provided cells are from callus obtained as described in the foregoing and/or suitably selected as described in the foregoing.

Preferably, the avocado cells are cultivated in the cell suspension culture for a time sufficient and under conditions sufficient to result in a lipid content of 0.5-10 wt-%, preferably 1-7 wt-%, more preferably 1.5-6 wt-%, even more preferably 2-5 wt-%, and/or a carbohydrate content of 10-65 wt-%, preferably 14-65 wt-%, more preferably 25-64 wt-%, even more preferably 55-64 wt-%, based on the total dry weight of the cultured avocado cells.

In certain preferred embodiments, the avocado cells are cultivated in the cell suspension culture for a time sufficient and under conditions sufficient to result in a content of polyunsaturated fatty acids, based on the total dry weight of the cultured avocado cells, at least 5 mg/g, preferably 5-25 mg/g, further preferably 7-20 mg/g, more preferably 10-18 mg/g, even more preferably 10-15 mg/g, and optionally a content of linoleic acid (C18:2n6) at least 5 mg/g, such as 5-20 mg/g, preferably at least 8 mg/g, more preferably 8-15 mg/g, even more preferably 8-10 mg/g, and/or of α-linoleic acid (C18:3n3) 1 mg/g or more, preferably 1.50 mg/g or more, more preferably 1-3 mg/g, even more preferably 1.5-2 mg/g. In certain embodiments, the avocado cells are cultivated in the cell suspension culture for a time and under conditions to result in a content of saturated fatty acids 3-15 mg/g, preferably 5-10 mg/g, and/or monounsaturated fatty acids less than 10 mg/g or less than 5 mg/g, preferably 1-3 mg/g, and optionally a content of oleic acid (C18:1) less than 5 mg/g, preferably at most 2 mg/g, based on the total dry weight of the cultured avocado cells.

In certain preferred embodiments, the avocado cells are cultivated in the cell suspension culture for a time sufficient and under conditions sufficient to result in a share of polyunsaturated fatty acids 30-70 wt-%, preferably 40-65 wt-%, more preferably 50-60 wt-%, and optionally a share of monounsaturated fatty acids 5-15 wt-%, preferably 8-12 wt-%, and/or a share of saturated fatty acids 20-40 wt-%, preferably 25-40 wt-%, more preferably 30-35 wt-%, based on the total weight of lipids in the cultured avocado cells. Optionally, the avocado cells are cultivated in the cell suspension culture for a time sufficient and under conditions sufficient to result in a share of 30-55 wt-%, preferably 35-50 wt-%, more preferably 40-50 wt-% linoleic acid (C18:2n-6), and/or 5-15 wt-% or 6-11 wt-% α-linoleic acid (C18:3n-3), and optionally 5-10 wt-% oleic acid (C18:1), and optionally 20-30 wt-% or 20-25 wt-% palmitic acid (C16:0), based on the total weight of lipids in the cultured avocado cells.

In certain preferred embodiments, the avocado cells are cultivated in the cell suspension culture for a time sufficient and under conditions sufficient to result in a content of sterols 0.5-2.0 mg/g, preferably 0.5-1.5 mg/g, more preferably 0.75-1.25 mg/g sterols, and optionally a content of β-sitosterol 0.5-1.0 mg/g in the cultured avocado cells, based on the total dry weight of the cultured avocado cells.

In certain preferred embodiments, the cultivation time and conditions of the cell suspension culture are selected to result in cultured avocado cells substantially (naturally) free from one or more, or even all, of 2-pentadecylfuran, 2-heptadecadienylfuran, 3-hexen-1-ol, caryophyllene oxide, trans-alpha-bergamotene, caryophyllene, and/or tocopherol, preferably free from at least caryophyllene and/or tocopherol, more preferably at least both.

In certain preferred embodiments, the conditions of the cell suspension culture are selected from one or more of:
a temperature within a range from 22° C. to 28° C., preferably from 24° C. to 26° C.;
elicitation with one or more of methyl jasmonate preferably in a concentration of 20-500 μM, jasmonic acid preferably in a concentration of 20-500 μM, ethylene preferably in a concentration of 50-200 mg/l, more preferably about 100 mg/l, ethephon preferably in a concentration of 50-200 mg/l, more preferably about 100 mg/l, salicylic acid, chitosan preferably in a concentration of 50-500 mg/l, more preferably about 200 mg/l, Zn ions, Cu ions, Fe ions, and/or Mn ions; and/or
a dark/light illumination regime 8 h/16 h dark/light, or darkness (without light periods). Such culture conditions may provide cultured avocado cells with desired compounds in desired amounts. Elicitation as described above may increase level(s) of flavor compound(s) (volatiles), colour, and/or phenolic(s) in the cultured avocado cells.

Cultivating the avocado cells in the cell suspension culture for a time (sufficient) and under conditions (sufficient) to result in certain content(s) of certain compound(s) may be a step of controlling production of said compound(s) in the cell suspension culture.

Preferably, the cultivation time in the cell suspension culture is within a range from 4 to 20 days, preferably from 7 to 15 days, more preferably from 7 to 12 days or from 7 to 11 days. It has been found that the duration of cell cultivation in the suspension culture may affect content of MUFAs, PUFAs, SAFAs, and sterols in the recovered or harvested cultured avocado cells. As shown in the present Examples, particularly high content of PUFAs were observed when the duration of cultivation in the cell suspension culture was within a range from 7 to 11 days. The amount of SAFAs in the cultured avocado cells seemed to follow a similar trend as the PUFAs, whereas higher content of MUFAs was observed at 4-7 days, after which the MUFAs content started to decrease. Increasing the cultivation time in the cell suspension culture increased the amount of sterols in the cultured avocado cells.

The cell suspension culture liquid medium may contain growth regulators. Preferably, the liquid medium contains at least one cytokinin or cytokinin-like plant growth regulator (CN) and at least one auxin (AUX). Examples of suitable CNs include kinetin, 6-(γ,γ-dimethylallylamino)purine, zeatin, or any combination thereof, and examples of suitable AUXs include α-naphthalene acetic acid, indole acetic acid, picloram, or any combination thereof, preferably α-naphthalene acetic acid, indole acetic acid, or any combination thereof. Preferably, the CN concentration is 0.1-5.0 ppm and the AUX concentration is 1.0-10 ppm.

The liquid medium may be Murashige and Skoog medium (MS-medium, as described in Murashige, T., and Skoog, F. (1962) A revised medium for rapid growth and bioassays with tobacco tissue cultures, Physiol. Plant. 15, 473-497), optionally comprising growth regulators, and/or Glu and Gly as described in the foregoing.

Preferably, the cell suspension culture is performed in an (ambient) air atmosphere.

In certain preferred embodiments, the liquid medium comprises:
at least one CN selected from kinetin, 6-(γ,γ-dimethylallylamino)purine, zeatin, or any combination thereof, and at least one AUX selected from α-naphthalene acetic acid, indole acetic acid, or any combination thereof, the CN concentration of the liquid medium preferably being 0.1-5.0 ppm and the AUX concentration of the liquid medium preferably being 1.0-10 ppm, and
glycine (Gly) and glutamic acid (Glu) preferably in a total amount of 1-15 mM, more preferably 0.01-0.05 mM Gly and 0.5-1.5 mM Glu.

In certain preferred embodiments, the cell suspension cultures are initiated with 0.03-0.07 g callus per g liquid medium in Murashige and Skoog medium (MS-medium, as described in Murashige, T., and Skoog, F. (1962) A revised medium for rapid growth and bioassays with tobacco tissue cultures, Physiol. Plant. 15, 473-497) with growth regulators, preferably at least one cytokinin or cytokinin-like plant growth regulator (CN) and at least one auxin (AUX), preferably kinetin preferably in an amount from 0.1-5.0 ppm, more preferably about 0.1 ppm and α-naphthalene acetic acid (NAA) preferably in an amount from 1.0 to 10 ppm, preferably about 1.0 ppm, at a temperature within a range from 22 to 28° C., such as at about 23° C., preferably on an orbital shaker at 90-120 rpm, preferably about 110 rpm, and preferably the cell suspension culture is performed in (ambient) air atmosphere, and optionally, the liquid medium comprises 0.01-0.05 mM Gly and 0.5-1.5 mM Glu.

The suspension culture may be subcultured, for example every 7-16 days, such as every 10 days. For subculturing, the biomass may be diluted for example by (about) 1:3 to 1:5 wt:wt biomass:liquid.

Preferably, the callus and the cultured avocado cells consists substantially of non-differentiated cells.

The present method may comprise a step of analysing avocado cells of the cell suspension culture, comprising obtaining avocado cells from the cell suspension culture, preferably drying the obtained avocado cells preferably by freeze drying, and subjecting the preferably dried avocado cells to analysis to determine content of certain compound(s). Examples of such compounds may be macronutrients, preferably at least proteins, more preferably at least proteins and lipids; polyunsaturated fatty acids, linoleic acid, and/or α-linoleic acid; and/or any other compound(s) discussed in the foregoing. The analysis step may be followed by, based on the determined content of the certain compound(s), deciding to either continue cultivation or harvest avocado cells. Such analysis step, preferably as followed by the determination step, are a mean to control content of certain compound(s) in the cultured avocado cells.

The avocado cells may be harvested from the cell suspension culture using any suitable harvesting method or technique. For examples, the method may comprise harvesting avocado cells from the cell suspension culture by vacuum filtration. Preferably, the harvested cells are washed to remove culture medium therefrom.

Optionally, the harvested and washed cultured avocado cells may be frozen, and optionally stored at a temperature within a range from −90° C. to −15° C., preferably within a range from −80° C. to −18° C., or the cultured avocado cells may be stored in liquid nitrogen (liquid N₂) at temperatures below −130° C., preferably below −140° C. Typically, the frozen cells are thawed, and optionally powdered and/or dried, before use as or in a food product.

Optionally, the obtained cultured avocado cells may be subjected to lysis, preferably with 0.7:1-3:1 wt/wt (wet weight) liquid to cultured avocado cells, more preferably about 1:1 wt/wt (wet weight) liquid to cultured avocado cells, to obtain cell lysate. The liquid may be water, such as sterile MilliQ water, or for example aqueous solution suitably additized. In other words, the cultured avocado cells may be in the form of a cell lysate. The cultured avocado cells subjected to lysis may have been stored frozen as described above and thawed, for example in an ice bath, prior to lysis. The cultured avocado cells subjected to lysis are typically provided without having subjected the cells to drying, i.e. the cultured avocado cells subjected to lysis have typically not been dried (freeze-dried). Providing the present cultured avocado cells as a cell lysate may have positive effect on mouth feel of the cells, for example they may be perceived to have a less sandy mouth feel, and/or providing the present cultured avocado cells as a cell lysate may have effect on processing of the cultured avocado cells, alone or with other food ingredients e.g. to form a food product, for example facilitating processing.

The cell lysate may be frozen, and optionally stored at a temperature within a range from −90° C. to −15° C., preferably within a range from −80° C. to −18° C., or the cell lysate may be stored in liquid nitrogen (liquid N₂) at temperatures below −130° C., preferably below −140° C. The frozen cell lysate may be thawed before use as or in a food product. In certain preferred embodiments, the method comprises powdering the cultured avocado cells, preferably comprising drying the cultured avocado cells preferably by freeze drying and/or spray drying, more preferably by freeze drying, optionally followed by mechanical powdering, such as homogenisation, grinding. and/or crushing. Freeze drying may provide a powder with an especially good or pleasant mouth feel. In certain embodiments, the cultured avocado cells are subjected to lysis followed by freeze drying. Such cells may have pleasant mouth feel.

The present cultured avocado cells may be powdered. Typically, said powdering comprises drying the cultured avocado cells and optionally subjecting the dried cells to mechanical powdering or homogenization.

Preferably, powdering the cultured avocado cells comprises freeze drying (lyophilization) and/or spray drying, optionally followed by mechanical powdering or homogenization. Simply drying, especially by freeze drying, the cultured avocado cells may suffice to powder them. However, additional powdering or homogenization of the powder may be employed where necessary or desired.

Mechanical powdering may comprise homogenisation, grinding, and/or crushing.

Preferably, the present method comprises using the cultured avocado cells in or as a food product for human consumption. Any chemicals and solvents used in the present method are preferably selected to yield cultured avocado cells suitable for use in food products for human consumption. As used herein, “suitable for use in food products for human consumption” does not necessarily indicate regulatory approval.

Herein is further provided a food product comprising cultured avocado cells as described herein. Preferably, the food product comprises, based on the total weight of the food product, 0.1-100 wt-%, further preferably 0.5-90 wt-%, more preferably 1.0-80 wt-%, even more preferably 5-70 wt-%, most preferably 10-65 wt-% of the present cultured avocado cells, preferably according to any one of the embodiments described in the foregoing, or obtainable or obtained with a method according to the present disclosure, preferably according to any one of the embodiments thereof.

The present cultured avocado cells may be used in or as a food product, the content of avocado cells in the food product preferably being as defined above. The cultured avocado cells may be used in the food product as obtained from the cell suspension culture after harvesting and optional washing (with or without freezing e.g. for storage purposes optionally followed by thawing), and/or the cultured avocado cells may be provided to the food product as a lysate, and/or as a powder preferably obtained as described in the foregoing. Preferably, the cultured avocado cells are provided to or included in the food product as a powder, more preferably a freeze dried or lyophilized powder.

The food product may for example be a nutritional supplement, such as protein powder, a spread, a dip, a guacamole, flakes, dumplings, squeezed biomass for sushi, a muesli, a granola, or a smoothie, including smoothie bowl.

Mesocarp of mature, fresh avocado fruit may be described as having a smooth and buttery and/or creamy texture, and a rich and nutty flavor. Mesocarp of immature avocado fruit may be considered to have a watery and firm texture, and a greasy taste.

The present cultured avocado cells do not have the shape and/or texture of the avocado fruit or its mature mesocarp as such. However, the cultured avocado cells may be formulated to obtain a texture resembling at least that of mashed, mature avocado mesocarp. Such formulation may include structural components, such as thickening agents, and optionally (added) water, especially if the cultured avocado cells are provided to the formulation as powder. These formulations may be used for example in guacamole, smoothie(s), spread(s), and/or dip(s). Accordingly, the present food product may comprise food-grade additives, preferably one or more of thickening agent(s), antioxidant(s), colourant(s), and/or flavorant(s). It has also been found that fresh cultured avocado cells as described herein have a pleasant flavour and odour, including avocado flavour attributes.

In certain embodiments, the food product comprises 1-5 wt-% thickening agent(s), and/or 1-4 wt-% antioxidant(s), based on the total weight of the food product.

To provide and/or maintain green or greenish, and/or yellow or yellowish colour resembling that of matured avocado mesocarp, colourant(s) and/or antioxidant(s) may be applied to formulations containing the present cultured avocado cells. For example, citric acid may be used to bleach brown and/or grey colour of the cultured avocado cells, and/or prevent formation thereof. For example, an aqueous solution extracted from kale leaves and/or spinach leaves, e.g. by immersion of optionally chopped leaves in water followed by filtration, may be provided as an aqueous colorant solution.

Preferably, the cultured avocado cells are used in or as a food product in the form of powder. Providing the cultured avocado cells as a powder may allow using them in existing processes and/or using existing equipment for avocado fruit powders. For examples, some commercial guacamoles are made using powdered avocado fruit mesocarp. In such processes and/or products powdered, cultured avocado cells could replace the avocado fruit powder in full or in part, even without investments and/or alterations in processes and/or equipment.

Implementation and embodiments of the present invention are further discussed in the following numbered clauses:
1. Cultured avocado cells comprising at least 15 wt-% proteins, based on the total dry weight of the cultured avocado cells.
2. The cultured avocado cells according to clause 1, comprising, based on the total dry weight of the cultured avocado cells,
   proteins 15-30 wt-%, preferably 20-30 wt-%, even more preferably 25-30 wt-%, and/or
   lipids 0.5-10 wt-%, preferably 1-7 wt-%, more preferably 1.5-6 wt-%, even more preferably 2-5 wt-%.
3. The cultured avocado cells according to clause 1 or 2, comprising polyunsaturated fatty acids, based on the total dry weight of the cultured avocado cells, at least 5 mg/g, preferably 5-25 mg/g, further preferably 7-20 mg/g, more preferably 10-18 mg/g, even more preferably 10-15 mg/g, and optionally the content of linoleic acid (C18:2n6) is at least 5 mg/g, such as 5-20 mg/g, preferably at least 8 mg/g, more preferably 8-15 mg/g, even more preferably 8-10 mg/g, and/or of α-linoleic acid (C18:3n3) is 1 mg/g or more, preferably 1.50 mg/g or more, more preferably 1-3 mg/g, even more preferably 1.5-2 mg/g, in the cultured avocado cells, based on the total dry weight of the cultured avocado cells.
4. The cultured avocado cells according to any one of the preceding clauses, comprising, based on the total dry weight of the cultured avocado cells, 3-15 mg/g, preferably 5-10 mg/g saturated fatty acids, and/or less than 10 mg/g, preferably less than 5 mg/g, more preferably 1-3 mg/g monounsaturated fatty acids, and optionally the content of oleic acid (C18:1) is less than 5 mg/g, preferably at most 2 mg/g, in the cultured avocado cells, based on the total dry weight of the cultured avocado cells.
5. The cultured avocado cells according to any one of the preceding clauses, comprising 10-65 wt-%, preferably 14-65 wt-%, more preferably 25-64 wt-%, even more preferably 55-64 wt-% carbohydrates, based on the total dry weight of the cultured avocado cells.
6. The cultured avocado cells according to any one of the preceding clauses, comprising, based on the total dry weight of the cultured avocado cells, 0.5-2.0 mg/g, preferably 0.5-1.5 mg/g, more preferably 0.75-1.25 mg/g sterols, and optionally the content of β-sitosterol is 0.5-1.0 mg/g in the cultured avocado cells, based on the total dry weight of the cultured avocado cells.
7. The cultured avocado cells according to any one of the preceding clauses, wherein the cultured avocado cells are substantially free from caryophyllene and/or tocopherol, and/or wherein the cultured avocado cells comprise catechin(s) and/or procyanidin(s), preferably catechin(s) and/or oligomeric procyanidin(s).
8. The cultured avocado cells according to any one of the preceding clauses, wherein the cultured avocado cells are in the form of a cell lysate or a powder, preferably a powder that is preferably obtained by freeze drying and/or spray drying, more preferably freeze drying, optionally followed by mechanical powdering, such as homogenisation, grinding and/or crushing.
9. The cultured avocado cells according to any one of the preceding clauses, wherein the cultured avocado cells are food grade avocado cells for human consumption.
10. A method for producing cultured avocado cells, the method comprising:
   providing avocado cells,
   initiating a cell suspension culture by inoculating the provided avocado cells into liquid medium and cultivating the avocado cells in the cell suspension culture for a time sufficient and under conditions sufficient to result in a protein content of at least 15 wt-%, based on the total dry weight of the avocado cells, and
   harvesting avocado cells from the cell suspension culture to obtain cultured avocado cells.
11. The method according to clause 10, wherein providing avocado cells comprises
   growing callus from avocado on solid growth medium, and
   providing avocado cells from the callus.
12. The method according to clause 10 or 11, wherein providing avocado cells comprises
   selecting avocado cells that produce polyunsaturated fatty acids in cell suspension culture, said avocado cells preferably being selected from the callus,
   providing the selected avocado cells, and
   wherein the cultivating in the cell suspension culture comprises
   cultivating for a time sufficient and under conditions sufficient to result in a polyunsaturated fatty acids content of at least 5 mg/g, based on the total dry weight of the cultured avocado cell.
13. The method according to any one of clauses 10-12, wherein the cultivation time and conditions of the cell suspension culture are selected to result in a lipid content of 0.5-10 wt-%, preferably 1-7 wt-%, more preferably 1.5-6 wt-%, even more preferably 2-5 wt-%, based on the total dry weight of the cultured avocado cells, and/or to obtain cultured avocado cells substantially free of caryophyllene and/or tocopherol.
14. The method according to any one of clauses 10-13, wherein the cultivation time in the cell suspension culture is within a range from 4 to 20 days, preferably from 7 to 15 days, more preferably from 7 to 12 days or from 7 to 11 days.
15. The method according to any one of clauses 10-14, wherein the conditions of the cell suspension culture are selected from one or more of:
   a temperature within a range from 22° C. to 28° C., preferably from 24° C. to 26° C.;
   elicitation with one or more of methyl jasmonate preferably in a concentration of 20-500 μM, jasmonic acid preferably in a concentration of 20-500 μM, ethylene preferably in a concentration of 50-200 mg/l, more preferably about 100 mg/l, ethephon preferably in a concentration of 50-200 mg/l, more preferably about 100 mg/l, salicylic acid, and/or chitosan preferably in a concentration of 50-500 mg/l, more preferably about 200 mg/l, Zn ions, Cu ions, Fe ions, and/or Mn ions; and/or
   a dark/light illumination regime 8 h/16 h dark/light, or darkness (without light periods).
16. The method according to any one of clauses 10-15, comprising using the cultured avocado cells in or as a food product for human consumption.
17. The method according to any one of clauses 10-16, comprising powdering the cultured avocado cells, preferably comprising drying the cultured avocado cells preferably by freeze drying and/or spray drying, more preferably by freeze drying, optionally followed by a mechanical powdering, such as homogenisation, grinding and/or crushing.
18. The method according to any one of clauses 10-17, wherein the callus and the cultured avocado cells consists substantially of non-differentiated cells.
19. The method according to any one of clauses 10-18, wherein the callus is grown from avocado fruit material or an avocado plant cut, preferably a cut or cuts from avocado seed, the seed optionally having been subjected to germination prior to cutting.
20. The method according to any one of the preceding clauses, comprising sterilizing the portion of the avocado from which callus is grown, prior to growing callus therefrom.
21. A food product comprising, based on the total weight of the food product, 0.1-100 wt-%, preferably 0.5-90 wt-%, further preferably 1.0-80 wt-%, more preferably 5-70 wt-%, even more preferably 10-65 wt-% cultured avocado cells according to any one of clauses 1-9.
22. The food product according to clause 21, comprising food-grade additives, preferably one or more of thickening agent(s), antioxidant(s), colourant(s), and/or flavorant(s).
23. The food product according to clause 21 or 22, comprising 1-5 wt-% thickening agent(s), and/or 1-4 wt-% antioxidant(s), based on the total weight of the food product.
24. The food product according to any one of clauses 21-23, wherein the food product is a nutritional supplement, a spread, a dip, a guacamole, flakes, dumplings, squeezed biomass for sushi, a muesli, a granola, or a smoothie.

### EXAMPLES

### Example 1—Callus Cultures

For establishing callus from avocado fruit, sterilized fruit material or cuts of obtained in vitro sterile plant were incubated on Murashige and Skoog medium (MS-medium, as described in Murashige, T., and Skoog, F. (1962). A revised medium for rapid growth and bioassays with tobacco tissue cultures, Physiol. Plant. 15, 473-497) with sucrose (3% w/v), plant growth regulators kinetin (0.1 ppm) and α-naphthalene acetic acid (NAA) (1.0 ppm), and solidified with agar (6.5 g/l). The incubation temperature for the samples was 22±2° C., and usually the samples were incubated both in dark and light. For illumination the day:night photoperiod (16:8 h) and normal light with irradiation 30-60 μmol/m²s were used. Obtained soft calli, usually formed within 7-21 days, were transferred on fresh medium.

MS-medium and illumination as described above were used for maintenance and cultivation of the obtained callus, but the temperature used was 24° C. The callus cultures were sub-cultured every 3 to 5 weeks.

### Example 1.1

Origin of avocado fruit: Chile, the mature avocado fruit was bought at local market in Finland.

The seed of the mature avocado fruit was germinated in water for 2 months, surface sterilized and cut into pieces. The pieces were incubated on the solid MS medium as described above. The callus culture obtained was separated to two lines maintained in light (Example 1.1V) and dark (Example 1.1P). The main colour of both callus lines is light grey, but occasionally Example 1.1V callus may turn partly to pink.

### Example 1.2

Origin of avocado fruit: Spain, the mature avocado fruit was bought at local market in Finland.

The seed of the mature avocado fruit was sterilized and planted in sterile MS-medium with no plant growth regulators favouring growth to in vitro plant. Cuts of the sterile stem of the obtained in vitro plant were incubated on MS medium with plant growth regulators as described above for establishment of callus. This callus was maintained in light, and parts of the light brown callus turned to pinkish after 3 to 6 weeks of incubation.

### Example 1.3

Origin of avocado fruit: Chile, Reed variety, the mature avocado fruit was bought at local market in Finland.

The material from inner layer of avocado fruit between fruit cover and mesocarp (pulp) was used for establishment of callus. The obtained callus line was maintained in light, and the colour of the callus was brownish but partly turned to pink after several weeks of incubation.

### Example 1.4

Origin of avocado fruit: Spain, Hass variety, the mature avocado fruit was bought at a local market in Finland.

The seed of the mature avocado fruit was surface sterilized, the embryo was revealed from the seed and used for establishment of callus culture. The callus culture obtained was maintained as two lines cultivated in light (Example 1.4V) and dark (Example 1.4P). The colour of the callus line Example 1.4P was light grey (almost white), and the line Example 1.4V turned pale pink after incubation for several weeks in light.

### Example 2—Cell Suspension Culture

The suspension cultures were initiated with 0.5-1 g well growing callus obtained similarly as described above in 15 mL MS-medium with kinetin (0.1 ppm) and NAA (1.0 ppm) in 50 mL Erlenmeyer flasks, at 23° C. on an orbital shaker at 110 rpm in air atmosphere. The cultures were subcultured regularly every 10 days and stepwise upscaled to 250 mL flasks containing 60 mL of culture. For subculturing the biomass was diluted 1:3 wt:wt biomass to liquid medium. Culturing was conducted either under 8 h/16 h dark/light illumination regime, or under dark.

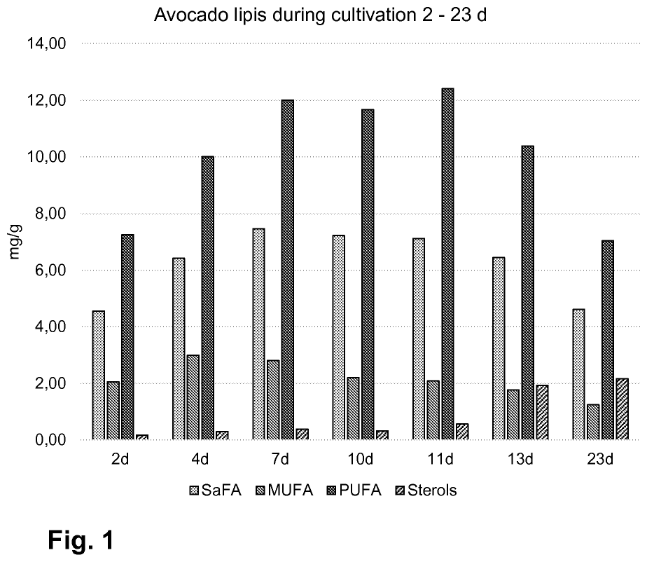

During cultivation in the suspension culture, content of fatty acids and sterols, namely contents of saturated fatty acids (SAFAs), monounsaturated fatty acids (MUFAs), and polyunsaturated fatty acids (PUFAs), and sterols, respectively, were analysed as a function of cultivation time. The results are shown in Table 1 and FIG. 1. The fatty acid contents were determined according to the method reported by Glaser et al. 2010 High-Throughput Analysis of Total Plasma Fatty Acid Composition with Direct In Situ Transesterification, PLOS ONE (https://doi.org/10.1371/journal.pone.0012045), sterol contents were determined according to the method described in Seppsnen-Laakso et al. 2017 UPLC-ELSD Analysis of Algal Lipid Classes and Derivatization of Bound and Free Fatty Acids and Sterols for GC-MS Methods, Biofuels from Algae, vol. 1980, pp. 223-232 (DOI: 10.1007/7651_2017_109).

TABLE 1
Contents (mg/g) of SAFAs, MUFAs, PUFAs, and sterols
in cultivated avocado cells according to the present
disclosure as function of cultivation time (days).

| | 2 d | 4 d | 7 d | 10 d | 11 d | 13 d | 23 d |
|---|---|---|---|---|---|---|---|
| SAFAs | 4.55 | 6.42 | 7.47 | 7.22 | 7.12 | 6.45 | 4.61 |
| MUFAs | 2.06 | 2.99 | 2.81 | 2.20 | 2.09 | 1.77 | 1.25 |
| PUFAs | 7.25 | 10.01 | 12.00 | 11.67 | 12.41 | 10.39 | 7.04 |
| Sterols | 0.18 | 0.29 | 0.39 | 0.33 | 0.56 | 1.93 | 2.17 |

As seen from Table 1 and FIG. 1, the PUFA content increased during the first week of cultivation, and highest PUFA contents were measured at 7-11 d cultivation times, after which PUFA content started to decrease. SAFAs followed a similar trend, although the change in SAFA content over the cultivation time was less pronounced compared to the PUFAs. MUFA content was at its highest at 4-7 d cultivation time, after which it started to decrease. The changes in MUFA content over cultivation time were small compared to PUFAs and SAFAs. The sterol content increased over the cultivation time of 2-23 d, and the highest sterol contents were measured at 23 d.

As seen from Table 1 and FIG. 1, the content of PUFAs was much higher than contents of MUFAs and SAFAs, respectively, at all measuring points 2 d-23 d.

Avocado cells were harvested from the cell suspension culture by vacuum filtration.

### Example 3—Analysis of Cultured Avocado Cells

### Example 3.1 Macronutrient Analysis

Cultured avocado cells according to the present disclosure were obtained from cell suspension culture conducted similarly as described in Example 2 and initiated from callus grown similarly as described in Example 1. The cultivation time in cell suspension culture was 11 days.

The macronutrient and energy contents were determined for the cultured avocado cells and compared to values reported for fresh avocado fruit. The results and analysis methods are reported in Table 1. Neither the cultured avocado cells nor the avocado fruit were dried prior to the analysis. Moisture content of the cultured avocado cells is reported in Table 1.

TABLE 1
Moisture, energy, and macronutrient contents of undried, cultured
avocado cells, and of avocado fruit, respectively.

| | Avocado cells from | Avocado fruit, |
|---|---|---|
| | Example 1.1P (frozen) | seedless (fresh fruit) |
| Lipids | <0.2 (±0.0) g/100 g | 19.4 g/100 g |
| FL15N FL Fat (foodstuff), Method: | | |
| NMKL 160 mod. Gravimetric | | |
| Moisture | 96.4 (±11.6) g/100 g | |
| FL033 FL Moisture (foodstuff, | | |
| feed) Method: NMKL 23: 1991, | | |
| mod. Gravimetric | | |
| Proteins | 1.0 (±0.1) g/100 g | 2.6 g/100 g |
| FL036 FL Protein (Kjeldahl) | | |
| Method: NMKL 6: 2003, mod. | | |
| Kjeldahl (titration) | | |
| Ash | 0.3 (±0.0) g/100 g | |
| FL037 FL Ash (foodstuff, feed) | | |
| Method: NMKL 173: 2005, mod. | | |
| Gravimetric | | |
| Carbohydrates | 2.2 g/100 g | 0.8 g/100 g |
| FL052 FL Carbohydrates | | |
| (computational) Computational | | |
| Energy | 13 kcal/100 g | 198 kcal/100 g |
| FL053 FL Energy content Method: | 55 kJ/100 g | 829 kJ/100 g |
| EU dir. 90/496/EY | | |
| Computational | | |

Already from Table 1 directly, it can be seen that the cultured avocado cells of this example has a lipid content way less and a clearly higher carbohydrate content than fresh avocado fruit. When accounting for the moisture, it can be concluded that protein content of the cultured avocado cells is very high, about 28 wt-%, based on the total dry weight of the avocado cells. This is clearly higher than for the fresh avocado fruit. For example, assuming a moisture content of approximately two thirds (w:w), the protein content of the fresh avocado fruit is just around 8 wt-%, based on the total dry weight of the fresh avocado fruit. Accordingly, the protein content of the cultured avocado cells of this example was more than threefold, about 3.5 fold, compared to the protein content of the fresh avocado fruit, based on respective dry weights.

### Example 3.2 Fatty Acids Analysis

Two samples of cultured avocado cells according to the present disclosure, C1 and C2, were obtained from cell suspension cultures conducted similarly as described in Example 2 and initiated from callus grown similarly as in Example 1. The cultivation time in cell suspension culture was 11 days. Before analyses, the cultured avocado cells were freeze dried, and analysis was conducted with the freeze dried cells.

Contents of fatty acids were determined according to the method reported by Glaser et al. 2010 High-Throughput Analysis of Total Plasma Fatty Acid Composition with Direct In Situ Transesterification, PLOS ONE (https://doi.org/10.1371/journal.pone.0012045), FAME, FFA, and sterol contents were determined according to the method described in Seppsnen-Laakso et al. 2017 UPLC-ELSD Analysis of Algal Lipid Classes and Derivatization of Bound and Free Fatty Acids and Sterols for GC-MS Methods, Biofuels from Algae, vol. 1980, pp. 223-232 (DOI: 10.1007/7651_2017_109).

As reference examples, two samples of mesocarp from matured avocado fruit, F1 and F2, were used. F1 and F2 were also freeze dried before analysis and analysis was conducted on freeze dried samples.

The results and calculated means are reported in Tables 2-4.

TABLE 2
Fatty acids of mesocarp of mature avocado fruit (F1, F2), and
of cultured avocado cells (C1, C2), respectively, expressed as
mg/g based on the total dry weight of the sample in question.

| | mg/g = g/kg | | | | | |
|---|---|---|---|---|---|---|
| | Fruit | | | Cells | | |
| Fatty acid | F1 | F2 | Mean | C1 | C2 | Mean |
| C14:0 Tetradecanoic acid (Myristic) | 0.04 | 0.00 | 0.00 | 0.02 | 0.02 | 0.02 |
| C15:0 Pentadecanoic acid | 0.00 | 0.00 | 0.00 | 0.05 | 0.05 | 0.05 |
| C16:0 Hexadecanoic acid (Palmitic) | 20.66 | 22.51 | 21.58 | 4.39 | 4.68 | 4.54 |
| C18:0 Octadecanoic acid (Stearic) | 0.63 | 0.69 | 0.66 | 0.85 | 0.90 | 0.88 |
| C20:0 Eicosanoic acid (Arachidic) | 0.08 | 0.09 | 0.09 | 0.13 | 0.14 | 0.14 |
| C22:0 Docosanoic acid (Behenic) | 0.00 | 0.00 | 0.00 | 0.28 | 0.30 | 0.29 |
| C23:0 Tricosanoic acid | 0.00 | 0.00 | 0.00 | 0.11 | 0.10 | 0.11 |
| C24:0 Tetracosanoic acid (Lignoceric) | 0.06 | 0.07 | 0.07 | 0.48 | 0.54 | 0.51 |
| SUM of Saturated | 21.48 | 30.10 | 30.10 | 6.33 | 6.74 | 6.54 |
| C16:1 9-Hexadecenoic acid | 7.50 | 8.28 | 7.89 | 0.00 | 0.00 | 0.00 |
| (Palmitoleic) | | | | | | |
| C18:1n9 Octadecenoic acid (Oleic) | 58.89 | 58.25 | 58.57 | 1.61 | 1.75 | 1.68 |
| C18:1n7 Octadecenoic acid (cis- | 8.45 | 9.50 | 8.98 | 0.07 | 0.09 | 0.08 |
| Vaccenic) | | | | | | |
| C20:1n9 Eicosenoic acid | 0.22 | 0.27 | 0.24 | 0.00 | 0.00 | 0.00 |
| SUM of Monounsaturated | 75.07 | 76.30 | 75.68 | 1.68 | 1.84 | 1.76 |
| C18:2n6 Octadecadienoic acid | 10.71 | 12.17 | 11.44 | 8.50 | 9.04 | 8.77 |
| (Linoleic) | | | | | | |
| C18:3n3 Octadecatrienoic acid (α- | 0.85 | 0.89 | 0.87 | 1.65 | 1.75 | 1.70 |
| Linolenic) | | | | | | |
| SUM of Polyunsaturated | 11.56 | 13.06 | 12.31 | 10.15 | 10.79 | 10.47 |
| C16:0 FFA | 0.20 | 0.16 | 0.18 | 0.15 | 0.16 | 0.15 |
| C18:1n9 FFA | 0.58 | 0.39 | 0.48 | 0.14 | 0.19 | 0.17 |
| C18:2n6 FFA | 0.00 | 0.00 | 0.00 | 0.08 | 0.10 | 0.09 |
| SUM of FFA | 0.78 | 0.55 | 0.66 | 0.38 | 0.45 | 0.41 |
| FAME | 108.10 | 112.76 | 110.43 | 18.16 | 19.38 | 18.77 |
| FFA | 0.78 | 0.55 | 0.66 | 0.38 | 0.45 | 0.41 |
| SUM ALL | 108.88 | 113.31 | 111.10 | 18.53 | 19.83 | 19.18 |
| Fat-% | 10.89 | 11.33 | 11.11 | 1.85 | 1.98 | 1.92 |

FAME refers to fatty acid methyl esters, FFA to free fatty acids.

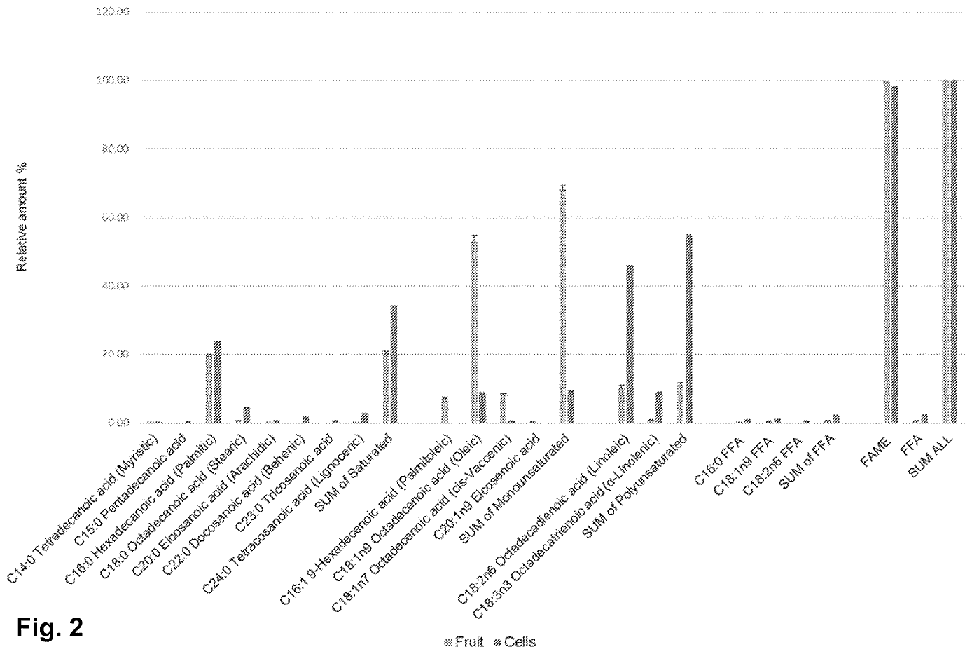

TABLE 3
Fatty acids of mesocarp of mature avocado fruit (F1, F2), and of cultured avocado cells
(C1, C2), respectively, expressed as %, based on the total weight of lipids in the
sample in question (relative amount). Results of Table 3 are shown in FIG. 2.

| | Relative amount (%) | | | | | |
|---|---|---|---|---|---|---|
| | Fruit | | | Cells | | |
| Fatty acid | F1 | F2 | Mean | C1 | C2 | Mean |
| C14:0 Tetradecanoic acid (Myristic) | 0.04 | 0.04 | 0.04 | 0.11 | 0.11 | 0.11 |
| C15:0 Pentadecanoic acid | 0.00 | 0.00 | 0.00 | 0.29 | 0.28 | 0.29 |
| C16:0 Hexadecanoic acid (Palmitic) | 18.97 | 19.86 | 19.42 | 23.70 | 23.61 | 23.66 |
| C18:0 Octadecanoic acid (Stearic) | 0.58 | 0.61 | 0.59 | 4.60 | 4.54 | 4.57 |
| C20:0 Eicosanoic acid (Arachidic) | 0.08 | 0.08 | 0.08 | 0.70 | 0.72 | 0.71 |
| C22:0 Docosanoic acid (Behenic) | 0.00 | 0.00 | 0.00 | 1.51 | 1.52 | 1.52 |
| C23:0 Tricosanoic acid | 0.00 | 0.00 | 0.00 | 0.62 | 0.51 | 0.57 |
| C24:0 Tetracosanoic acid (Lignoceric) | 0.06 | 0.06 | 0.06 | 2.61 | 2.71 | 2.66 |
| SUM of Saturated | 19.72 | 20.66 | 20.19 | 34.15 | 34.00 | 34.08 |
| C16:1 9-Hexadecenoic acid (Palmitoleic) | 6.89 | 7.31 | 7.10 | 0.00 | 0.00 | 0.00 |
| C18:1n9 Octadecenoic acid (Oleic) | 54.09 | 51.40 | 52.75 | 8.69 | 8.83 | 8.76 |
| C18:1n7 Octadecenoic acid (cis- | 7.76 | 8.39 | 8.07 | 0.37 | 0.47 | 0.42 |
| Vaccenic) | | | | | | |
| C20:1n9 Eicosenoic acid | 0.20 | 0.23 | 0.22 | 0.00 | 0.00 | 0.00 |
| SUM of Monounsaturated | 68.94 | 67.33 | 68.14 | 9.06 | 9.30 | 9.18 |
| C18:2n6 Octadecadienoic acid (Linoleic) | 9.84 | 10.74 | 10.29 | 45.86 | 45.60 | 45.73 |
| C18:3n3 Octadecatrienoic acid (α- | 0.78 | 0.79 | 0.79 | 8.90 | 8.84 | 8.87 |
| Linolenic) | | | | | | |
| SUM of Polyunsaturated | 10.62 | 11.53 | 11.07 | 54.75 | 54.44 | 54.60 |
| C16:0 FFA | 0.19 | 0.14 | 0.16 | 0.83 | 0.78 | 0.81 |
| C18:1n9 FFA | 0.53 | 0.34 | 0.44 | 0.78 | 0.98 | 0.88 |
| C18:2n6 FFA | 0.00 | 0.00 | 0.00 | 0.42 | 0.49 | 0.46 |
| SUM of FFA | 0.71 | 0.48 | 0.60 | 2.04 | 2.25 | 2.14 |
| FAME | 99.29 | 99.52 | 99.40 | 97.96 | 97.75 | 97.86 |
| FFA | 0.71 | 0.48 | 0.60 | 2.04 | 2.25 | 2.14 |
| SUM ALL | 100.00 | 100.00 | 100.00 | 100.00 | 100.00 | 100.00 |

FAME refers to fatty acid ester, FFA to free fatty acids.

As seen from Tables 2 and 3, and FIG. 2, the fatty acids profiles of C1 and C2 differ from those of F1 and F2. For example, C1 and C2 contained linoleic acid (and α-linolic acid) as main fatty acid(s) (FA), while F1 and F2 contained oleic acid as its main FA. As seen from Table 3 and FIG. 2, the share of PUFAs of the total lipids was much higher in C1 and C2 than in F1 and F2, while the share of MUFAs was lower and the share of SAFAs higher in C1 and C2 compared to F1 and F2. PUFAs are generally considered to be particularly desired FAs as they may provide health benefits. Although SAFAs may be regarded as less healthy compared to unsaturated fatty acids, due to the much lower total lipid content of C1 and C2, the content of SAFAs, expressed as mg/g based in the total dry weight of the respective sample, was in fact much lower in C1 and C2 compared to F1 and F2, even more than three or even more than 4 times less, as seen in Table 2.

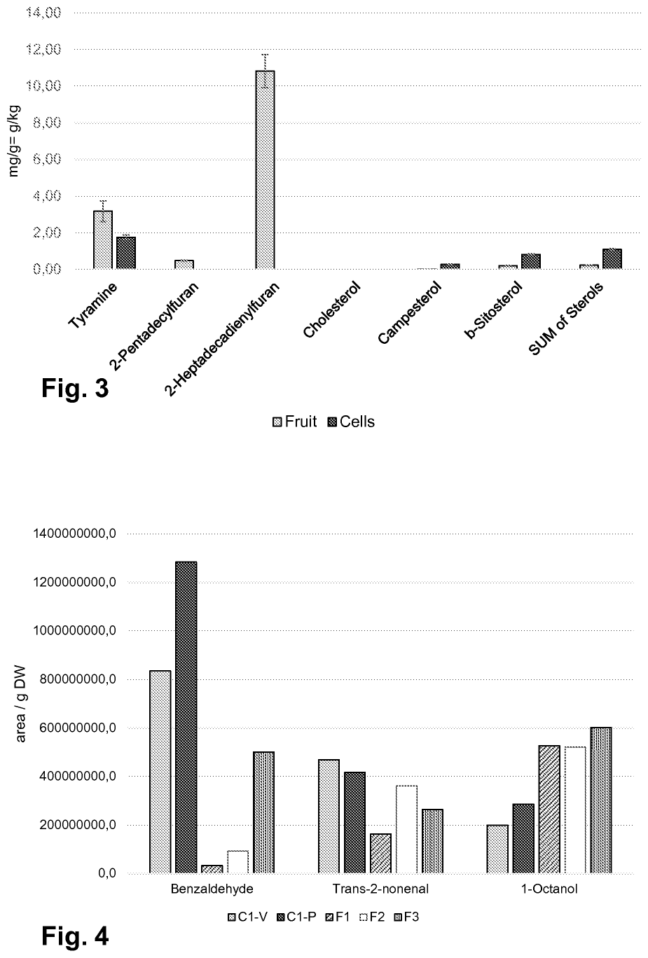

Content of sterols, as well as of tyramine, 2-pentadecylfuran, and 2-heptadecadienylfuran, as measured according to Seppsnen-Laakso et al. 2017 UPLC-ELSD Analysis of Algal Lipid Classes and Derivatization of Bound and Free Fatty Acids and Sterols for GC-MS Methods, Biofuels from Algae, vol. 1980, pp. 223-232 (DOI: 10.1007/7651_2017_109), are reported in Table 4 and in FIG. 3.

TABLE 4
Sterols of mesocarp of mature avocado fruit (F1, F2), and of cultured
avocado cells (C1, C2), respectively, expressed as mg/g based on
the total dry weight of the sample in question. Content of tyramine,
2-pentadecylfuran, and 2-heptadecadienylfuran, respectively, are
also reported in Table 4 as mg/g based on the total dry weight of
the sample in question. Results of Table 4 are shown in FIG. 3.

| | mg/g = g/kg | | | | | |
|---|---|---|---|---|---|---|
| | Fruit | | | Cells | | |
| Compound | F1 | F2 | Mean | C1 | C2 | Mean |
| Tyramine | 3.58 | 2.78 | 3.18 | 1.66 | 1.84 | 1.75 |
| 2-Pentadecylfuran | 0.47 | 0.49 | 0.48 | 0.00 | 0.00 | 0.00 |
| 2-Heptadec- | 10.19 | 11.47 | 10.83 | 0.00 | 0.00 | 0.00 |
| adienylfuran | | | | | | |
| Cholesterol | 0.005 | 0.003 | 0.004 | 0.002 | 0.002 | 0.002 |
| Campesterol | 0.02 | 0.03 | 0.03 | 0.26 | 0.29 | 0.27 |
| β-Sitosterol | 0.21 | 0.18 | 0.20 | 0.80 | 0.83 | 0.81 |
| SUM of Sterols | 0.240 | 0.218 | 0.229 | 1.057 | 1.118 | 1.087 |

As seen from Table 4 and FIG. 3, C1 and C2 contained more sterols than F1 and F2, the sterol content being four fold or more, even five fold or even more, based on respective dry weights. Especially the content of β-sitosterol is higher in C1 and C2 compared to F1 and F2, based on respective dry weights.

### Example 3.3 Analysis of Volatiles

Two samples of cultured avocado cells according to the present disclosure, C1-V and C1-P, were obtained from cell suspension culture conducted similarly as described in Example 2 and initiated from callus grown in Example 1.1V (C1-V) and Example 1.1P (C1-P), respectively. In the suspension culture, C1-V was cultivated under 8 h/16 h dark/light illumination regime, and C1-P was cultivated in dark.

Before analyses, the cultured avocado cells were freeze dried, and analysis was conducted with the freeze dried cells. The analysis method is described in Example 3.0 below.

As reference examples, three samples of mesocarp from matured avocado fruit, F1, F2, and F3, were used. F1, F2, and F3 were freeze dried before analysis, and analysis was conducted on freeze dried samples as described in Example 3.0.

The results are reported in Tables 5-12 and FIGS. 4-12.

TABLE 5
Analysed contents of benzaldehyde, trans-2-nonenal, and 1-octanol
of freeze dried, cultured avocado cells (C1-V and C1-P), and
of freeze dried avocado fruit mesocarp, respectively, expressed
as area/g based on the total dry weight of the sample in question.
Results of Table 5 are shown in FIG. 4

| Sample | Benzaldehyde | Trans-2-nonenal | 1-Octanol |
|---|---|---|---|
| C1-V | 834489610.8 | 468672021.9 | 199510859.4 |
| C1-P | 1283511710.6 | 416202021.4 | 285862448.1 |
| F1 | 32561023.7 | 162627159.4 | 526857362.1 |
| F2 | 93153848.5 | 361542015.8 | 521437990.7 |
| F3 | 500427214.6 | 264509448.4 | 602029334.4 |

As seen from Table 5 and FIG. 4, C1-P and C1-V contained much more benzaldehyde than F1-F3. C1-V and C1-P contained slightly more trans-2-nonenal, and clearly less 1-octenal than F1-F3.

TABLE 6
Analysed contents of 1-hexanol, 3-hexen-1-ol, tetradecane, and
3-octen-2-one of freeze dried, cultured avocado cells (C1-V and
C1-P), and of freeze dried avocado fruit mesocarp, respectively,
expressed as area/g based on the total dry weight of the sample
in question. Results of Table 6 are shown in FIG. 5.

| Sample | 1-Hexanol | 3-Hexen-1-ol | Tetradecane | 3-Octen-2-one |
|---|---|---|---|---|
| C1-V | 226221958.6 | 0.0 | 72623437.0 | 193097942.9 |
| C1-P | 169673431.0 | 0.0 | 136555474.3 | 221568752.3 |
| F1 | 692983349.2 | 4682585.2 | 159380954.9 | 301269896.4 |
| F2 | 1077593396.3 | 0.0 | 317036304.3 | 270159700.8 |
| F3 | 532575400.2 | 0.0 | 418664781.9 | 352527944.0 |

As seen from Table 6 and from FIG. 5, C1-V and C1-P contained much less 1-hexanol than F1-F3. Tetradecane and 3-octen-2-one contents, respectively, were somewhat lower for C1-V and C1-P than F1-F3.

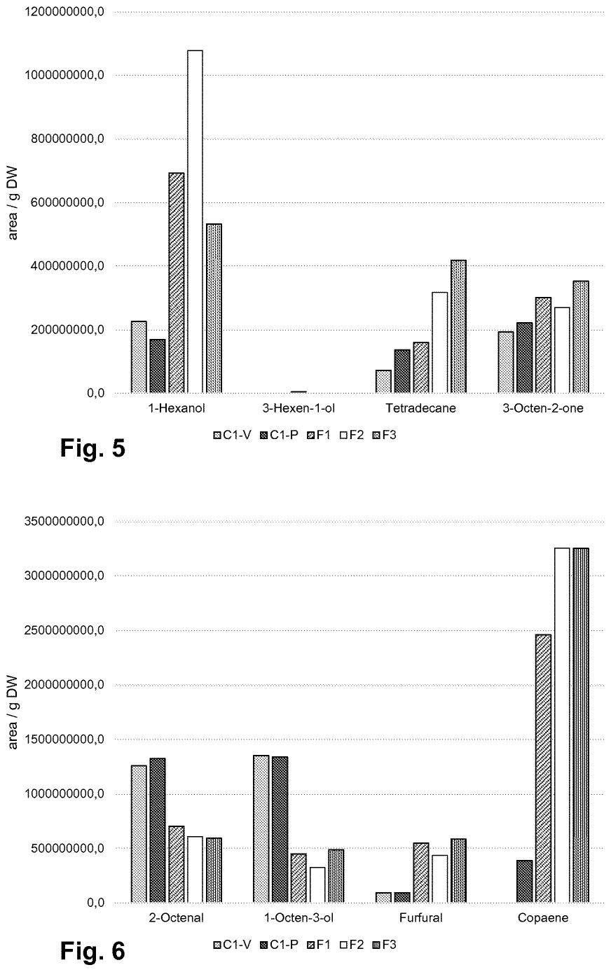

TABLE 7
Analysed contents of 2-octenal, 1-octen-3-ol, furfural, and
copaene of freeze dried, cultured avocado cells (C1-V and C1-
P), and of freeze dried avocado fruit mesocarp, respectively,
expressed as area/g based on the total dry weight of the sample
in question. Results of Table 7 are shown in FIG. 6.

| Sample | 2-Octenal | 1-Octen-3-ol | Furfural | Copaene |
|---|---|---|---|---|
| C1-V | 1259076952.8 | 1352797139.7 | 92847886.9 | 0.0 |
| C1-P | 1325115206.6 | 1339402569.9 | 95346906.3 | 389716155.5 |
| F1 | 704084561.8 | 451744970.7 | 549141147.9 | 2460992826.4 |
| F2 | 607827685.5 | 324666471.3 | 436185661.7 | 3254997760.2 |
| F3 | 595220848.9 | 488970704.1 | 586532092.7 | 3252907491.3 |

As seen in Table 7 and FIG. 6, C1-V and C1-P contained more 2-octenal and 1-octen-3-ol, respectively, and less furfural and much less copaene than F1-F3.

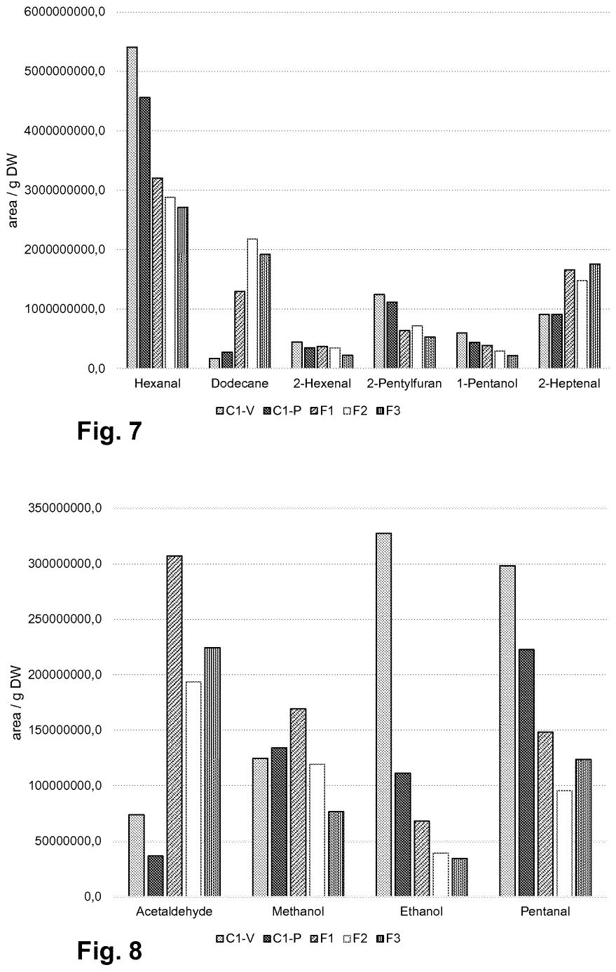

TABLE 8
Analysed contents of hexanal, dodecane, 2-hexenal, 2-pentylfuran, 1-pentanol,
and 2-heptanal of freeze dried, cultured avocado cells (C1-V and C1-P), and of
freeze dried avocado fruit mesocarp, expressed as area/g based on the total dry
weight of the sample in question. Results of Table 8 are shown in FIG. 7.

| Sample | Hexanal | Dodecane | 2-Hexenal | 2-Pentylfuran | 1-Pentanol | 2-Heptenal |
|---|---|---|---|---|---|---|
| C1-V | 5405480720.6 | 172381286.2 | 448552839.2 | 1247484293.8 | 601082057.6 | 912349447.4 |
| C1-P | 4556714779.4 | 276378576.8 | 351094220.6 | 1115255797.4 | 442804967.5 | 915005774.9 |
| F1 | 3204085250.0 | 1297914457.6 | 374954564.3 | 638532543.7 | 391572233.7 | 1660132304.8 |
| F2 | 2884641271.3 | 2181566188.0 | 347690556.7 | 721816267.6 | 297113910.8 | 1484636561.0 |
| F3 | 2712482608.2 | 1920283853.3 | 224849897.5 | 535235027.9 | 220161964.3 | 1758329276.9 |

As seen in Table 8 and in FIG. 7, C1-V and C1-P contained clearly more hexanal, and much less dodecane than F1-F3. The contents of 2-hexenal and 1-pentanol were relatively similar in C1-V, C1-P, and F1-F3, whereas F1-F3 contained more 2-heptenal and less 2-pentylfuran than C1-V and C1-P.

TABLE 9
Analysed contents of acetaldehyde, methanol, ethanol, and pentanal
of freeze dried, cultured avocado cells (C1-V and C1-P), and
of freeze dried avocado fruit mesocarp, respectively, expressed
as area/g based on the total dry weight of the sample in question.
Results of Table 9 are shown in FIG. 8.

| Sample | Acetaldehyde | Methanol | Ethanol | Pentanal |
|---|---|---|---|---|
| C1-V | 73821216.0 | 124616642.0 | 327352859.6 | 298278682.5 |
| C1-P | 36683045.6 | 133955801.4 | 111306005.2 | 222748536.6 |
| F1 | 306972368.6 | 169257115.6 | 68124571.7 | 148340965.5 |
| F2 | 193594737.8 | 119366591.0 | 39351372.4 | 95598132.4 |
| F3 | 224319275.1 | 76556113.1 | 34440864.6 | 123715694.9 |

As seen in Table 9 and FIG. 8, the content of acetaldehyde was much lower in C1-V and C1-P than in F1-F3, methanol content was on a somewhat similar level, whereas ethanol and pentanal contents, respectively, where higher in C1-V and C1-P compared to F1-F3. Ethanol content was particularly high in C1-V, which might indicate that light:dark regime affects ethanol content.

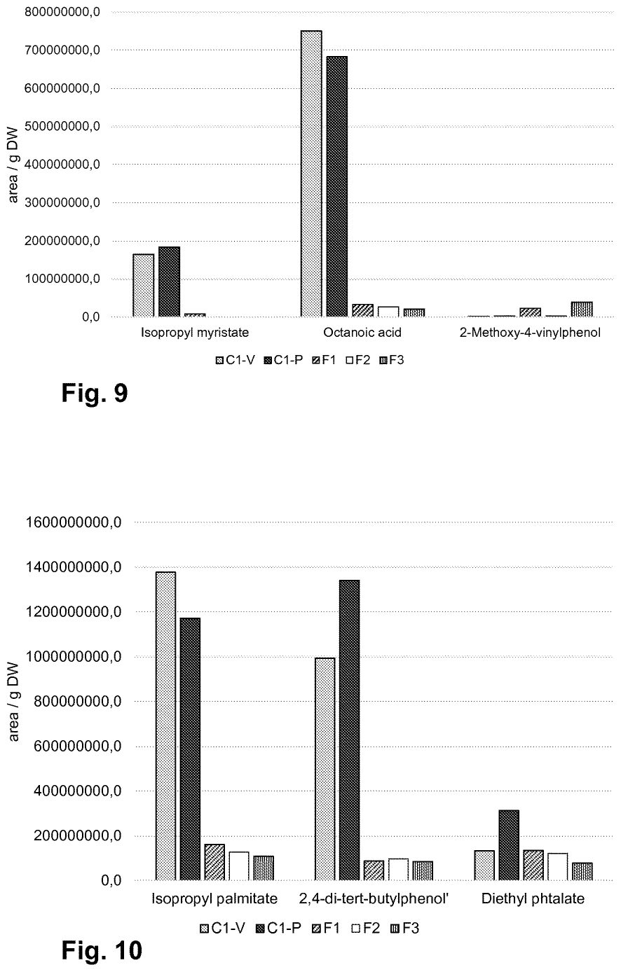

TABLE 10
Analysed contents of isopropyl myristate, octanoic acid, and
2-methoxy-4-vinylphenol of freeze dried, cultured avocado cells
(C1-V and C1-P), and of freeze dried avocado fruit mesocarp,
expressed as area/g based on the total dry weight of the sample
in question. Results of Table 10 are shown in FIG. 9

| | Isopropyl | | 2-Methoxy-4- |
|---|---|---|---|
| Sample | myristate | Octanoic acid | vinylphenol |
| C1-V | 164216429.6 | 750597120.7 | 2469228.8 |
| C1-P | 183539151.3 | 682624050.9 | 3418688.1 |
| F1 | 9138738.3 | 32886689.8 | 22959216.0 |
| F2 | 0.0 | 27029316.5 | 3488354.3 |
| F3 | 0.0 | 20944973.1 | 39153084.3 |

As seen in Table 10 and in FIG. 9, the contents of isopropyl myristate and octanoic acid, respectively, where much higher in C1-V and C1-P than in F1-F3. For F2 and F3 no isopropyl myristate content was even reported. C1-V, C1-P, and F1-F3 contained quite similar, seemingly small amounts of 2-methoxy-4-vinylphenol.

TABLE 11
Analysed contents of isopropyl palmitate, 2,4-di-tert-butylphenol,
and diethyl phtalate, of freeze dried, cultured avocado cells
(C1-V and C1-P), and of freeze dried avocado fruit mesocarp,
expressed as area/g based on the total dry weight of the sample
in question. Results of Table 11 are shown in FIG. 10.

| | Isopropyl | 2,4-di-tert- | |
|---|---|---|---|
| Sample | palmitate | butylphenol′ | Diethyl phtalate |
| C1-V | 1377132472.1 | 992304775.5 | 134168204.5 |
| C1-P | 1171658126.7 | 1339911570.9 | 313917082.7 |
| F1 | 162012054.9 | 87680776.7 | 135565220.7 |
| F2 | 128277671.3 | 97847160.9 | 122452067.6 |
| F3 | 109420017.7 | 84846151.7 | 78960100.1 |

As seen in Table 11 and FIG. 10, C1-V and C1-P contained much more isopropyl palmitate and 2,4-di-tert-butylphenol, respectively, than F1-F3. Diethyl phtalate content was at rather similar level for the studied samples C1-V, C1-P, and F1-F3.

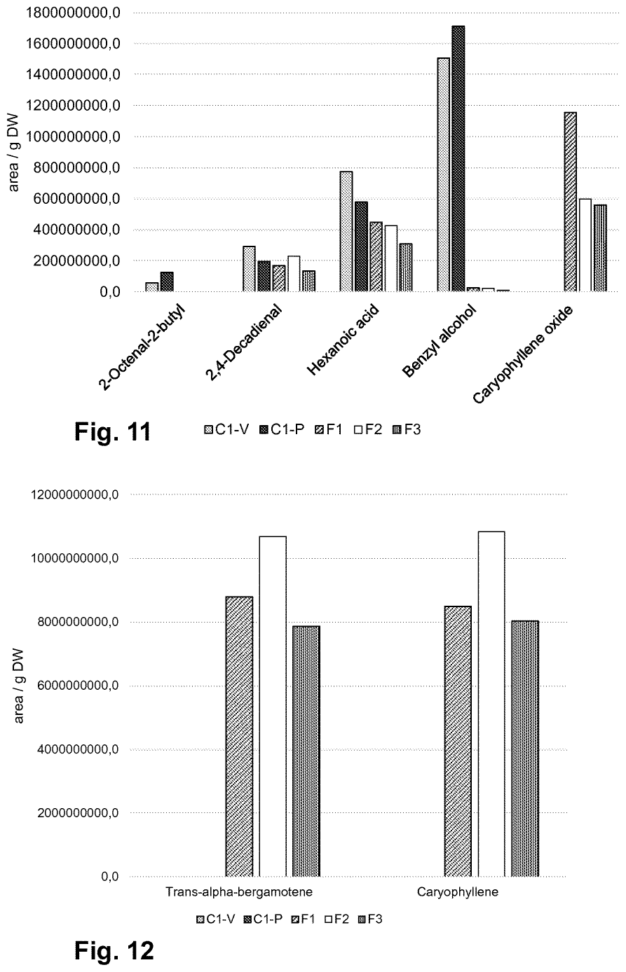

TABLE 12
Analysed contents of 2-octenal-2-butyl, 2,4-decadienal, hexanoic acid,
benzyl alcohol, and caryophyllene oxide, of freeze dried, cultured
avocado cells (C1-V and C1-P), and of freeze dried avocado fruit mesocarp
(F1-F3), expressed as area/g based on the total dry weight of the
sample in question. Results of Table 12 are shown in FIG. 11.

| | 2-Octenal-2- | 2,4- | Hexanoic | Benzyl | Caryophyllene |
|---|---|---|---|---|---|
| Sample | butyl | Decadienal | acid | alcohol | oxide |
| C1-V | 57348434.2 | 291481322.0 | 773891153.0 | 1505907329.5 | 0.0 |
| C1-P | 125337922.5 | 195960453.0 | 578692407.5 | 1712057175.6 | 0.0 |
| F1 | 0.0 | 168333048.9 | 448521155.6 | 26652966.5 | 1154807124.0 |
| F2 | 0.0 | 229461871.2 | 426646391.0 | 22814272.3 | 596766088.0 |
| F3 | 0.0 | 134946699.4 | 308406769.9 | 8149939.7 | 560112019.7 |

As seen in Table 12 and FIG. 11, content of 2-octenal-2-butyl was clearly higher for C1-V and C1-P compared to F1-F3, for which no 2-octenal-2-butyl content was reported. C1-V and C1-P were both without caryophyllene oxide content reported. Caryophyllene oxide was however present in F1-F3 in clearly non-negligible amounts. C1-V and C1-P contained much more benzyl alcohol compared to F1-F3 for which just very low amounts were reported. C1-V and C1-P had a slightly higher content of hexanoic acid than F1-F3, content of 2,4-decadienal was somewhat similar between the studied samples C1-V, C1-P, and F1-F3.

FIG. 12 shows analysed contents of trans-alpha-bergamotene, and caryophyllene of freeze dried, cultured avocado cells (C1-V and C1-P), and of freeze dried avocado fruit mesocarp (F1-F3). As seen in FIG. 12, C1-V and C1-P were substantially without trans-alpha-bergamotene, and caryophyllene content, although the contents in F1-F3 seemed to be rather high.

### Example 3.0 Analysis Method of Volatiles

Organic compounds (volatiles) were determined from freeze dried cultured avocado cells according to the present disclosure and freeze dried mesocarp of mature avocado fruit.

For volatile compound analyses 100 mg of sample material was accurately weighed into pre-heated (70° C., 1 h) headspace vial. Milli-Q water (1 ml) was added and the vial was closed with pre-heated magnetic cork. GC-MS analyses were performed with Agilent HP-Innowax 19091N-1361 (60 m×250 μm×0.25 μm) column, Agilent 7890B oven and 5977B mass chromatography attached with CTC/PAL Autosampler. SPME (solid phase microextraction) fibre used was 1 cm Supelco 57329-U DVB/CAR/PDMS. Samples were incubated at 80° C. (1 min) and the fibre was then kept in headspace vial for 30 min with agitation (250 rpm, 20 s on, 10 s off). The fibre was desorpted in the GC-oven inlet for 6 min. Between the samples the fibre was conditioned (10 min, 270° C.). Analyses were performed in splitless mode (250° C.), with 1.2 ml/ml flow rate. The temperature program used was 40° C. for 3 min, 10° C./min to 240° C., and hold for 7 min. MS detection was performed in range 25-600 amu.

### Example 3.1—Analysis of Catechins and Procyanidins

Samples of cultured avocado cells according to the present disclosure, C1-V, C1-P1, C1-P2, C2-V. and C3-V, were obtained from cell suspension cultures conducted similarly as described in Example 2 and initiated from callus grown in Example 1.1V (C1-V), Example 1.1P (C1-P1, C1-P2), Example 1.2 (C2-V), and Example 1.4V (C3-V), respectively.

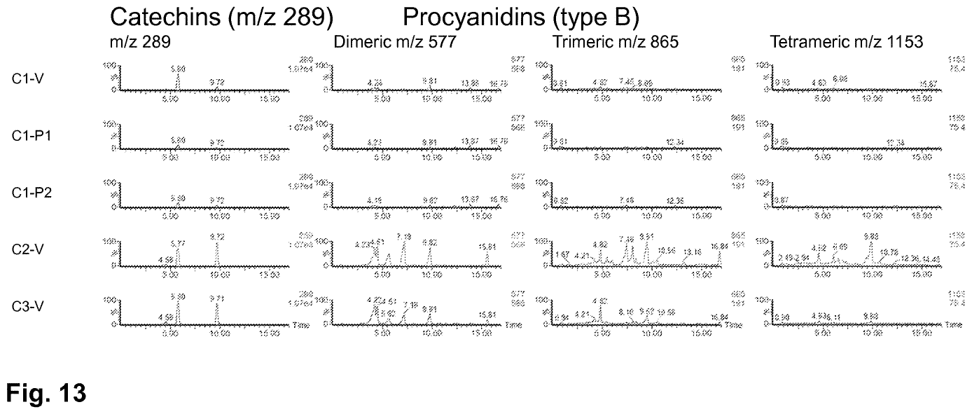

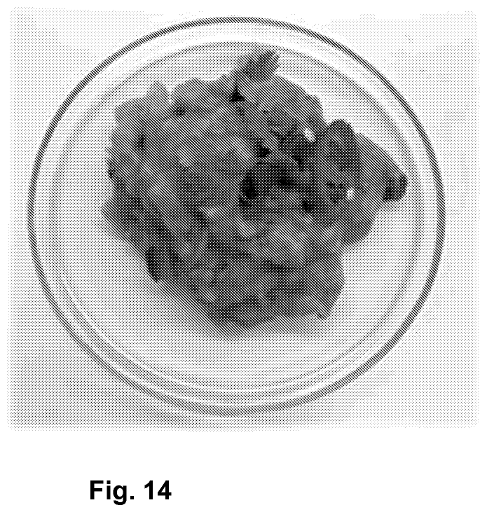

Qualitative analysis of catechins and procyanidins was performed from 10 mg samples as methanolic extracts with UPLC-DAD-Qtof-MS, and identifications of compounds (polyphenols) were done according to Rosero et al., Analysis of Phenolic Composition of Byproducts (Seeds and Peels) of Avocado (Persea americana Mill.) Cultivated in Colombia, Molecules 2019, 24(17), 3209; (https://doi.org/10.3390/molecules24173209). The results are shown in FIG. 13.

As seen in FIG. 13, all of the studied samples C1-V, C1-P1, C1-P2, C2-V, and C3-V showed at least some catechin and procyanidin peaks, indicating presence thereof. Most and largest peaks were obtained for C2-V and C3-V, especially C2-V. So there seems to be some catechin(s) and/or procyanidins, even oligomeric procyanidins, present in C1-V, C1-P1, C1-P2, C2-V, and C3-V, at least in C2-V and C3-V.

### Example 4—Food Product Comprising Cultured Avocado Cells

Guacamole type food products according to the present disclosure were formed using cultured avocado cells stored frozen and thawed before use.

The cultured avocado cells were obtained from suspension cultures conducted similarly as described in Example 2 and initiated from callus grown similarly as in Example 1.

The cultured avocado cells were formulated as described below.

Food grade thickening agents, namely Psyllium 0.4 wt-% (Virtasalmen Viljat) and xantane 2 wt-% (Farina; solution made in Puhdistamo's MCT-oil), were added to and mixed with the cultured avocado cells to provide a mixture having desired structure resembling mashed avocado fruit mesocarp.

2.5 wt-% citric acid was included in the mixture as an antioxidant and to bleach the brownish colour.

The above wt-% s are based on the total weight of the mixture.

Aqueous kale leaf extract was then blended with the mixture as a colorant in an amount of 30 ml to 100 g avocado cells. Said kale leaf extract was obtained as follows: kale leaves (30 g in 70 ml water, room temperature) were pounded with pamix for ca. one minute, centrifuged at 4000 rpm for 20 min, and supernatant was recovered as the kale leaf extract.

The above formulation comprising cultured avocado cells was then mixed with chili, onion, tomato, and coriander to obtain guacamole.

FIG. 14 shows a photograph of the guacamole formed using the formulation comprising (intact) cultured avocado cells. As seen in FIG. 14, said guacamole comprising cultured avocado cells resembles to its appearance and texture very much conventional guacamole made from avocado fruit mesocarp.

### Example 5—Cell Suspension Culture with Amino Acid Feeding

The cultured avocado cells were obtained from suspension cultures conducted similarly as described in Example 2 and initiated from callus grown similarly as in Example 1, but with the following modifications: Avocado cell culture was propagated in flasks (250 ml Erlenmeyer, 70 ml working volume) and combined suspensions were divided in 100 ml Erlenmeyer flasks (20 ml working volume) at day 5. Glycine and glutamic acid were dissolved in water, filter sterilized and added in the cell suspension cultures with final concentrations of 0.03 mM and 1 mM, respectively. No additions were made to control cultures. After feeding, cultivation was continued normally, until 10 days from the start. Cells were collected, biomass was measured and cells were freeze-dried before protein analyses.

The protein analysis (total amino acid composition and content) was carried out as follows: Reagents and Materials: AccQ-Tag reagent kit, Mass TRAK™ Amino Acid Analysis concentrate A and eluent B were obtained from Waters (Milford, MA, USA). Amino Acid Standard Solution, Amino Acid Standards Physiological, Basics, L-isoleusine, glutamine and norvaline were obtained from Sigma-Aldrich (St. Luis, Missouri, USA).

For the determination of total amino acid content the samples (5 mg, accurately weighed) were first oxidized with fresh performic acid solution (0.5 ml) prepared by mixing 5 ml of 30% hydrogen peroxide (Sigma-Aldrich) with 45 ml of formic acid-phenol solution (88% formic acid and 0.5% phenol). After incubation at 0° C. for 12 h ice-cold H₂O (1 ml) was added and the mixture was evaporated to dryness under nitrogen flow. Subsequently, 1 ml of 6 N HCl containing 0.1% phenol was added and the samples were hydrolysed at 110° C. for 20 h. After hydrolysis samples were evaporated to dryness, reconstituted in 1000 μl of H₂O and further diluted 1/10. Derivatization was performed according to Waters (Milford, MA, USA) AccQ-Tag method.

For the determination of tryptophan, samples were hydrolysed in alkaline conditions with 4 M lithium hydroxide (LiOH) solution containing ascorbic acid (95 mM). The lyophilized samples (2 mg, accurately weighed) were dissolved in 0.9 ml of freshly prepared hydrolysis solution. A vacuum was applied to the hydrolysis tubes followed by nitrogen replacement to the headspace. The samples were hydrolysed at 110° C. for 16 h. After hydrolysis, samples were neutralized by adding 600 ml of hydrochloric acid (6 M), methyl-tryptophan internal standard was added and samples were derivatized as described above.

UPLC analysis: Analysis was performed on an Acquity UPLC system, Waters (Milford, MA, USA) with diode array detector. Chromatography was performed using an Acquity Mass TRAK™ (2.1×100 mm, 1.7 μm) column, Waters (Milford, USA), kept at 43° C. Injection volume was 1 μL. Separation was performed at a flow rate of 0.4 mL/min using a gradient elution program. Signal was detected at 260 nm. Standards were derivatized as the samples.

His, Ser, Arg Gly, Asp, Glu, Thr, Ala, Pro, Cys, Lys, Tyr, Met, Val, lie, Leu, Phe and Trp were quantified in the samples. In acid hydrolysis Asn is converted to Asp and Gln to Glu. Cysteine and methionine were determined as cysteic acid and methionine sulfone after the oxidation procedure. Protein content of the samples can be estimated based on the sum of the determined amino acids.

Protein contents of the cultured avocado cells with and without Gly+Glu feeding, as determined according to the analysis method hereabove, are reported in Table 13. Control refers to the avocado cells cultured without Gly+Glu feeding.

TABLE 13
Total amino acids (protein content) and biomass. Results are presented
as mean of three biological replicates ± standard deviation.

| | Total amino acids (mg/g DW) | Biomass (g/L DW) |
|---|---|---|
| Gly + Glu feeding | 203.2 (5.5) | 6.69 (0.09) |
| Control | 165.9 (10.8) | 7.96 (0.32) |

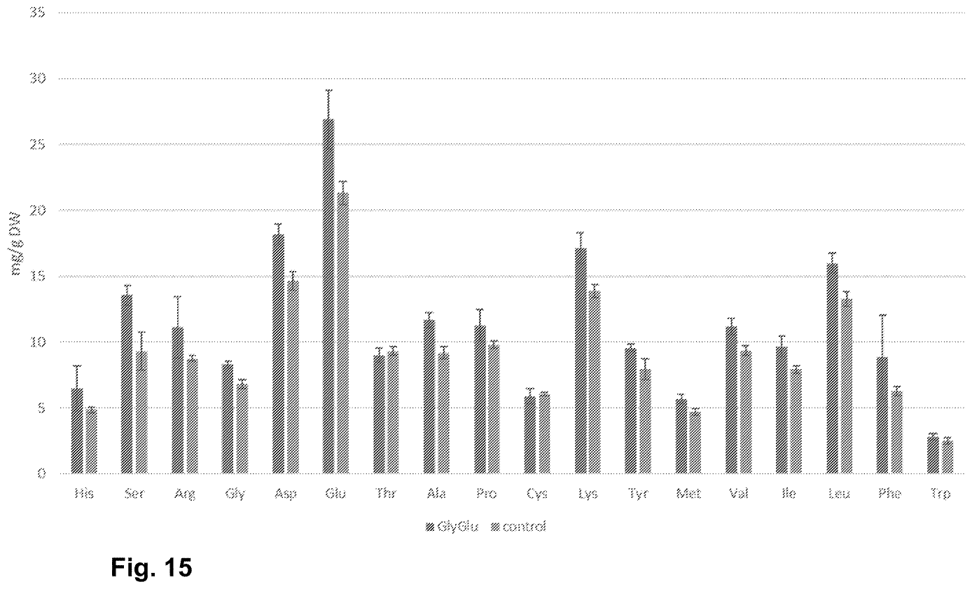

A total increase of 22% (p<0.01, T-test) in total amino acid content with Gly+Glu feeding was observed compared to the control cultures without the Gly+Glu feeding, as seen in Table 13. That is, the recovered avocado cells from the Gly+Glu fed culture had a 22% higher protein content compared to the avocado cells from the control culture. This increase in protein content is especially beneficially when use is made of the whole cultured avocado cells, without extraction of compounds. FIG. 15 shows analysed individual amino acid contents of the cultured avocado cells with and without Gly+Glu feeding. FIG. 15 shows that in the avocado cells cultured with Gly+Glu feeding all amino acids increased compared to or stayed at the level of the control culture without Gly+Glu feeding.

Gly+Glu feeding caused a 16% reduction in the recovered biomass. However, even if accounting for the biomass reduction, feeding with Gly+Glu resulted in a total 3% increase in protein yield. However, if using whole cultured avocado cells, the protein content of the recovered biomass might be seen as a more interesting features than total protein yield.

### Example 6—Sensory Evaluation of Cultured Avocado Cells

Avocado cells were cultivated for 12 days in shake flasks similarly as described in Examples 1 and 2, filtered with miracloth and washed with sterile water. The recovered, cultured avocado cells were stored in a freezer. A portion of the cultured avocado cells was treated with microfluidisation, i.e. lysed.

Microfluidisation or lysis was executed in food grade facilities. The buckets and tools used in the lysis as well as the microfluidizer were cleaned according to a careful cleaning procedure. The vessels were first cleaned with dishwashing liquid and tap water. Then both the vessels and the microfluidizer were cleaned with ethanol and further washed thoroughly with reverse osmosis (RO) water before starting the microfluidisation. A dispersion was prepared from thawed cultured avocado cells by using purified reverse osmosis (RO) water. The thawed cultured avocado cell mass was diluted with the RO water in a 50:50 w:w ratio. The sample size was 115 g. The cell disruption (lysis) was carried out by feeding the dispersion after pre-mixing into the microfluidizer, a Microfluidics fluidizer M110-EH with two Z-type chambers. The pre-mixing was carried out by dispersion using a Waring blender for approximately 15 seconds, or the sample was pre-mixed in the microfluidizer by running it once through a 400 μm chamber of the microfluidizer at a 400 bar operating pressure. In the actual cell disruption phase, the samples were run once through a chamber of the microfluidizer having a diameter of 400 μm at a 1500 bar operating pressure. The output samples were collected in clean sample containers.

Undried cultured avocado cells as well as lysed, undried avocado cells were evaluated for their odour and flavour attributes, respectively, using generic descriptive analysis. Also, mouthfeel of cultured avocado cell samples was studied. The evaluations were performed in a sensory laboratory by trained panelists.

Lexicon generation session took place with a small panel of 4 panelists, and panel training session took place with 8 panelists. Products or samples were evaluated in duplicate (2 sessions—1 h apart), with 8 panelists in each session. The assessment was carried out on a 0-10 linear numeric scale. Reference samples were tasted before evaluating the samples to tie the scale.

Table 14 reports odour and flavour attributes obtained for undried (fresh) cultured avocado cells as well as lysed, undried (fresh) avocado cells.

TABLE 14
Odour and flavour attributes of undried, cultured avocado
cells, and lysed, undried cultured avocado cells.

| | Odour | Flavour |
|---|---|---|
| Avocado | sweet (mild), | melon (2); watermelon (3); green (2-4); |
| cells, | melon (2) | cucumber (6); avocado (3); unripe avocado |
| fresh | | (1.5); woody (mild); chemical (1); sweet (2); |
| | | bitter (4) |
| Avocado | sweet (mild), | melon (2.5); watermelon (1-3); green/grassy |
| cells, | melon (3); | (1-3); cucumber (1-3); unripe avocado (3.5); |
| fresh, | strawberry | woody (mild); sweet (2); bitter aftertaste (2) |
| lysed | kissel (1) | |

As can be deduced from Table 14 above, the fresh cultured avocado cells and lysed, fresh cultured avocado cells had both pleasant odour and flavour attributes, including avocado flavour attributes. Based on the sensory evaluation of mouthfeel, it was observed that freeze-drying significantly improved the mouthfeel of the cells. Freeze-drying reduced undesired sensory attributes, such as graininess (presence of small, granular particles, resembling the texture of sand in the mouth), perceptibility (the ease with which the sandy mouthfeel can be detected), and particle size (the size of the granules or particles present in the product), and increased desired sensory attribute uniformity (the consistency of the sandiness throughout the product, whether it is clustered in certain areas or evenly distributed) in a statistically significant extent compared to corresponding fresh (undried) cells, for example comparing lysed fresh cells with lysed freeze dried cells.

Various embodiments have been presented. It should be appreciated that in this document, words comprise, include, and contain are each used as open-ended expressions with no intended exclusivity.

The foregoing description has provided by way of non-limiting examples of particular implementations and embodiments a full and informative description of the best mode presently contemplated by the inventors for carrying out the invention. It is however clear to a person skilled in the art that the invention is not restricted to details of the embodiments presented in the foregoing, but that it can be implemented in other embodiments using equivalent means or in different combinations of embodiments without deviating from the characteristics of the invention.

Furthermore, some of the features of the afore-disclosed example embodiments may be used to advantage without the corresponding use of other features. As such, the foregoing description shall be considered as merely illustrative of the principles of the present invention, and not in limitation thereof. Hence, the scope of the invention is only restricted by the appended patent claims.

1. Cultured avocado cells comprising at least 15 wt-% proteins, based on the total dry weight of the cultured avocado cells.

2. The cultured avocado cells according to claim 1, comprising, based on the total dry weight of the cultured avocado cells,
proteins 15-30 wt-%, and/or
lipids 0.5-10 wt-%.

3. The cultured avocado cells according to claim 1 comprising at least 5 mg/g polyunsaturated fatty acids, in the cultured avocado cells, based on the total dry weight of the cultured avocado cells.

4. The cultured avocado cells according to claim 1, comprising, based on the total dry weight of the cultured avocado cells, 3-15 mg/g saturated fatty acids, and/or less than 10 mg/g monounsaturated fatty acids.

5. The cultured avocado cells according to claim 1, comprising 10-65 wt-% carbohydrates, based on the total dry weight of the cultured avocado cells.

6. The cultured avocado cells according to claim 1, comprising, based on the total dry weight of the cultured avocado cells, 0.5-2.0 mg/g sterols.

7. (canceled)

8. The cultured avocado cells according to claim 1, wherein the cultured avocado cells are in the form of a cell lysate or a powder.

9. (canceled)

10. A method for producing cultured avocado cells, the method comprising:
providing avocado cells,
initiating a cell suspension culture by inoculating the provided avocado cells into liquid medium and cultivating the avocado cells in the cell suspension culture for a time sufficient and under conditions sufficient to result in a protein content of at least 15 wt-%, based on the total dry weight of the avocado cells, and
harvesting avocado cells from the cell suspension culture to obtain cultured avocado cells.

11. The method according to claim 10, wherein the liquid medium comprises
at least one cytokinin or cytokinin-like plant growth regulator (CN) selected from kinetin, 6-(γ,γ-dimethylallylamino)purine, zeatin, or any combination thereof, and
at least one auxin (AUX) selected from α-naphthalene acetic acid, indole acetic acid, or any combination thereof.

12. (canceled)

13. The method according to claim 10, wherein the liquid medium comprises glycine (Gly) and glutamic acid (Glu).

14. The method according to claim 10, wherein providing avocado cells comprises
growing callus from avocado on solid growth medium, and
providing avocado cells from the callus.

15. The method according to claim 10 wherein providing avocado cells comprises
selecting avocado cells that produce polyunsaturated fatty acids in cell suspension culture, said avocado cells,
providing the selected avocado cells, and
wherein the cultivating in the cell suspension culture comprises
cultivating for a time sufficient and under conditions sufficient to result in a polyunsaturated fatty acids content of at least 5 mg/g, based on the total dry weight of the cultured avocado cell.

16. The method according to claim 10, wherein the cultivation time and conditions of the cell suspension culture are selected to result in a lipid content of 0.5-10 wt-% based on the total dry weight of the cultured avocado cells.

17. The method according to claim 10 wherein the cultivation time in the cell suspension culture is within a range from 4 to 20 days.

18. (canceled)

19. (canceled)

20. The method according to claim 10, wherein the callus and the cultured avocado cells consists substantially of non-differentiated cells.

21. The method according to claim 10, wherein the callus is grown from:
a. avocado fruit material,
b. an avocado plant cut, or
c. cuts from avocado seed.

22. A food product comprising cultured avocado cells according to claim 1.

23. The food product according to claim 22, comprising food-grade additives.

24. (canceled)

25. The food product according to claim 22, wherein the food product is a nutritional supplement, a spread, a dip, a guacamole, flakes, dumplings, squeezed biomass for sushi, a muesli, a granola, or a smoothie.

26. The cultured avocado cells according to claim 1, comprising the content of linoleic acid (C18:2n6) is at least 5 mg/g in the cultured avocado cells, based on the total dry weight of the cultured avocado cells.
